SEQUENCE LISTING (8 sequences)

SEQ ID NO: 1 - protein, 831 aa, Homo sapien
LDNPKCIDRSKPSEPIQPVNTWEISSLPSSTVTSETLPSSLSPSVEKYRYQDEDTPPQEHISPQITNEVIGPELVHVSEKNLSEIENVHGFVSHSHISPIKPTEAVLPSPPTVPVIPVLPVPAENTVILPTIPQANPPPVLVNTDSLETPTYVNGTDADYEYEEITLERGNSGLGFSIAGGTDNPHIGDDSSIFITKIITGGAAAQDGRLRVNDCILRVNEVDVRDVTHSKAVEALKEAGSIVRLYVKRRKPVSEKIMEIKLIKGPKGLGFSIAGGVGNQHIPGDNSIYVTKIIEGGAAHKDGKLQIGDKLLAVNNVCLEEVTHEEAVTALKNTSDFVYLKVAKPTSMYMNDGYAPPDITNSSSQPVDNHVSPSSFLGQTPASPARYSPVSKAVLGDDEITREPRKVVLHRGSTGLGFNIVGGEDGEGIFISFILAGGPADLSGELRKGDRIISVNSVDLRAASHEQAAAALKNAGQAVTIVAQYRPEEYSRFEAKIHDLREQMMNSSISSGSGSLRTSQKRSLYVRALFDYDKTKDSGLPSQGLNFKFGDILHVINASDDEWWQARQVTPDGESDEVGVIPSKRRVEKKERARLKTVKFNSKTRDKGEIPDDMGSKGLKRGQEEYVLSYEPVNQQEVNYTRPVIILGPMKDRINDDLISEFPDKFGSCVPHTTRPKRDYEVDGRDYHFVTSREQMEKDIQEHKFIEAGQYNNHLYGTSVQSVREVAEKGKHCILDVSGNAIKRLQIAQLYPISIFIKPKSMENIMEMNKRLTEEQARKTFERAMKLEQEFTEHFTAIVQGDTLEDIYNQVKQIIEEQSGSYIWVPAKEKL

SEQ ID NO: 2 - protein, 857 aa, Homo sapien
LDNPKCIDRSKPSEPIQPVNTWEISSLPSSTVTSETLPSSLSPSVEKYRYQDEDTPPQEHISPQITNEVIGPELVHVSEKNLSEIENVHGFVSHSHISPIKPTEAVLPSPPTVPVIPVLPVPAENTVILPTIPQANPPPVLVNTDSLETPTYVNGTDADYEYEEITLERGNSGLGFSIAGGTDNPHIGDDSSIFITKIITGGAAAQDGRLRVNDCILRVNEVDVRDVTHSKAVEALKEAGSIVRLYVKRRKPVSEKIMEIKLIKGPKGLGFSIAGGVGNQHIPGDNSIYVTKIIEGGAAHKDGKLQIGDKLLAVNNVCLEEVTHEEAVTALKNTSDFVYLKVAKPTSMYMNDGYAPPDITNSSSQPVDNHVSPSSFLGQTPASPARYSPVSKAVLGDDEITREPRKVVLHRGSTGLGFNIVGGEDGEGIFISFILAGGPADLSGELRKGDRIISVNSVDLRAASHEQAAAALKNAGQAVTIVAQYRPEEYSRFEAKIHDLREQMMNSSISSGSGSLRTSQKRSLYVRALFDYDKTKDSGLPSQGLNFKFGDILHVINASDDEWWQARQVTPDGESDEVGVIPSKRRVEKKERARLKTVKFNSKTRDKGEIPDDMGSKGLKHVTSNASDSESSYLILITDEYGCSKGRGQEEYVLSYEPVNQQEVNYTRPVIILGPMKDRINDDLISEFPDKFGSCVPHTTRPKRDYEVDGRDYHFVTSREQMEKDIQEHKFIEAGQYNNHLYGTSVQSVREVAEKGKHCILDVSGNAIKRLQIAQLYPISIFIKPKSMENIMEMNKRLTEEQARKTFERAMKLEQEFTEHFTAIVQGDTLEDIYNQVKQIIEEQSGSYIWVPAKEKL

SEQ ID NO: 3 - protein, 844 aa, Homo sapien
LDNPKCIDRSKPSEPIQPVNTWEISSLPSSTVTSETLPSSLSPSVEKYRYQDEDTPPQEHISPQITNEVIGPELVHVSEKNLSEIENVHGFVSHSHISPIKPTEAVLPSPPTVPVIPVLPVPAENTVILPTIPQANPPPVLVNTDSLETPTYVNGTDADYEYEEITLERGNSGLGFSIAGGTDNPHIGDDSSIFITKIITGGAAAQDGRLRVNDCILRVNEVDVRDVTHSKAVEALKEAGSIVRLYVKRRKPVSEKIMEIKLIKGPKGLGFSIAGGVGNQHIPGDNSIYVTKIIEGGAAHKDGKLQIGDKLLAVNNVCLEEVTHEEAVTALKNTSDFVYLKVAKPTSMYMNDGYAPPDITNSSSQPVDNHVSPSSFLGQTPASPARYSPVSKAVLGDDEITREPRKVVLHRGSTGLGFNIVGGEDGEGIFISFILAGGPADLSGELRKGDRIISVNSVDLRAASHEQAAAALKNAGQAVTIVAQYRPEEYSRFEAKIHDLREQMMNSSISSGSGSLRTSQKRSLYVRALFDYDKTKDSGLPSQGLNFKFGDILHVINASDDEWWQARQVTPDGESDEVGVIPSKRRVEKKERARLKTVKFNSKTRDKGEIPDDMGSKGLKHVTSNASDSESSYRGQEEYVLSYEPVNQQEVNYTRPVIILGPMKDRINDDLISEFPDKFGSCVPHTTRPKRDYEVDGRDYHFVTSREQMEKDIQEHKFIEAGQYNNHLYGTSVQSVREVAEKGKHCILDVSGNAIKRLQIAQLYPISIFIKPKSMENIMEMNKRLTEEQARKTFERAMKLEQEFTEHFTAIVQGDTLEDIYNQVKQIIEEQSGSYIWVPAKEKL

SEQ ID NO: 4 - protein, 793 aa, Homo sapien
LDNPKCIDRSKPSEPIQPVNTWEISSLPSSTVTSETLPSSLSPSVEKYRYQDEDTPPQEHISPQITNEVIGPELVHVSEKNLSEIENVHGFVSHSHISPIKVNGTDADYEYEEITLERGNSGLGFSIAGGTDNPHIGDDSSIFITKIITGGAAAQDGRLRVNDCILRVNEVDVRDVTHSKAVEALKEAGSIVRLYVKRRKPVSEKIMEIKLIKGPKGLGFSIAGGVGNQHIPGDNSIYVTKIIEGGAAHKDGKLQIGDKLLAVNNVCLEEVTHEEAVTALKNTSDFVYLKVAKPTSMYMNDGYAPPDITNSSSQPVDNHVSPSSFLGQTPASPARYSPVSKAVLGDDEITREPRKVVLHRGSTGLGFNIVGGEDGEGIFISFILAGGPADLSGELRKGDRIISVNSVDLRAASHEQAAAALKNAGQAVTIVAQYRPEEYSRFEAKIHDLREQMMNSSISSGSGSLRTSQKRSLYVRALFDYDKTKDSGLPSQGLNFKFGDILHVINASDDEWWQARQVTPDGESDEVGVIPSKRRVEKKERARLKTVKFNSKTRDKGEIPDDMGSKGLKHVTSNASDSESSYRGQEEYVLSYEPVNQQEVNYTRPVIILGPMKDRINDDLISEFPDKFGSCVPHTTRPKRDYEVDGRDYHFVTSREQMEKDIQEHKFIEAGQYNNHLYGTSVQSVREVAEKGKHCILDVSGNAIKRLQIAQLYPISIFIKPKSMENIMEMNKRLTEEQARKTFERAMKLEQEFTEHFTAIVQGDTLEDIYNQVKQIIEEQSGSYIWVPAKEKL

SEQ ID NO: 5 - protein, 811 aa, Homo sapien
LDNPKCIDRSKPSEPIQPVNTWEISSLPSSTVTSETLPSSLSPSVEKYRYQDEDTPPQEHISPQITNEVIGPELVHVSEKNLSEIENVHGFVSHSHISPIKANPPPVLVNTDSLETPTYVNGTDADYEYEEITLERGNSGLGFSIAGGTDNPHIGDDSSIFITKIITGGAAAQDGRLRVNDCILRVNEVDVRDVTHSKAVEALKEAGSIVRLYVKRRKPVSEKIMEIKLIKGPKGLGFSIAGGVGNQHIPGDNSIYVTKIIEGGAAHKDGKLQIGDKLLAVNNVCLEEVTHEEAVTALKNTSDFVYLKVAKPTSMYMNDGYAPPDITNSSSQPVDNHVSPSSFLGQTPASPARYSPVSKAVLGDDEITREPRKVVLHRGSTGLGFNIVGGEDGEGIFISFILAGGPADLSGELRKGDRIISVNSVDLRAASHEQAAAALKNAGQAVTIVAQYRPEEYSRFEAKIHDLREQMMNSSISSGSGSLRTSQKRSLYVRALFDYDKTKDSGLPSQGLNFKFGDILHVINASDDEWWQARQVTPDGESDEVGVIPSKRRVEKKERARLKTVKFNSKTRDKGEIPDDMGSKGLKHVTSNASDSESSYRGQEEYVLSYEPVNQQEVNYTRPVIILGPMKDRINDDLISEFPDKFGSCVPHTTRPKRDYEVDGRDYHFVTSREQMEKDIQEHKFIEAGQYNNHLYGTSVQSVREVAEKGKHCILDVSGNAIKRLQIAQLYPISIFIKPKSMENIMEMNKRLTEEQARKTFERAMKLEQEFTEHFTAIVQGDTLEDIYNQVKQIIEEQSGSYIWVPAKEKL

SEQ ID NO: 6 - protein, 866 aa, Homo sapien
LDNPKCIDRSKPSEPIQPVNTWEISSLPSSTVTSETLPSSLSPSVEKYRYQDEDTPPQEHISPQITNEVIGPELVHVSEKNLSEIENVHGFVSHSHISPIKPTEAVLPSPPTVPVIPVLPVPAENTVILPTIPQANPPPVLVNTDSLETPTYVNGTDADYEYEEITLERGNSGLGFSIAGGTDNPHIGDDSSIFITKIITGGAAAQDGRLRVNDCILRVNEVDVRDVTHSKAVEALKEAGSIVRLYVKRRKPVSEKIMEIKLIKGPKGLGFSIAGGVGNQHIPGDNSIYVTKIIEGGAAHKDGKLQIGDKLLAVNNVCLEEVTHEEAVTALKNTSDFVYLKVAKPTSMYMNDGYAPPDITNSSSQPVDNHVSPSSFLGQTPASPARYSPVSKAVLGDDEITREPRKVVLHRGSTGLGFNIVGGEDGEGIFISFILAGGPADLSGELRKGDRIISVNSVDLRAASHEQAAAALKNAGQAVTIVAQYRPEEYSRFEAKIHDLREQMMNSSISSGSGSLRTSQKRSLYVRALFDYDKTKDSGLPSQGLNFKFGDILHVINASDDEWWQARQVTPDGESDEVGVIPSKRRVEKKERARLKTVKFNSKTRDKGQSFNDKRKKNLFSRKFPFYKNKDQSEQETSDADQHVTSNASDSESSYRGQEEYVLSYEPVNQQEVNYTRPVIILGPMKDRINDDLISEFPDKFGSCVPHTTRPKRDYEVDGRDYHFVTSREQMEKDIQEHKFIEAGQYNNHLYGTSVQSVREVAEKGKHCILDVSGNAIKRLQIAQLYPISIFIKPKSMENIMEMNKRLTEEQARKTFERAMKLEQEFTEHFTAIVQGDTLEDIYNQVKQIIEEQSGSYIWVPAKEKL

SEQ ID NO: 7 - protein, 833 aa, Homo sapien
LDNPKCIDRSKPSEPIQPVNTWEISSLPSSTVTSETLPSSLSPSVEKYRYQDEDTPPQEHISPQITNEVIGPELVHVSEKNLSEIENVHGFVSHSHISPIKANPPPVLVNTDSLETPTYVNGTDADYEYEEITLERGNSGLGFSIAGGTDNPHIGDDSSIFITKIITGGAAAQDGRLRVNDCILRVNEVDVRDVTHSKAVEALKEAGSIVRLYVKRRKPVSEKIMEIKLIKGPKGLGFSIAGGVGNQHIPGDNSIYVTKIIEGGAAHKDGKLQIGDKLLAVNNVCLEEVTHEEAVTALKNTSDFVYLKVAKPTSMYMNDGYAPPDITNSSSQPVDNHVSPSSFLGQTPASPARYSPVSKAVLGDDEITREPRKVVLHRGSTGLGFNIVGGEDGEGIFISFILAGGPADLSGELRKGDRIISVNSVDLRAASHEQAAAALKNAGQAVTIVAQYRPEEYSRFEAKIHDLREQMMNSSISSGSGSLRTSQKRSLYVRALFDYDKTKDSGLPSQGLNFKFGDILHVINASDDEWWQARQVTPDGESDEVGVIPSKRRVEKKERARLKTVKFNSKTRDKGQSFNDKRKKNLFSRKFPFYKNKDQSEQETSDADQHVTSNASDSESSYRGQEEYVLSYEPVNQQEVNYTRPVIILGPMKDRINDDLISEFPDKFGSCVPHTTRPKRDYEVDGRDYHFVTSREQMEKDIQEHKFIEAGQYNNHLYGTSVQSVREVAEKGKHCILDVSGNAIKRLQIAQLYPISIFIKPKSMENIMEMNKRLTEEQARKTFERAMKLEQEFTEHFTAIVQGDTLEDIYNQVKQIIEEQSGSYIWVPAKEKL

SEQ ID NO: 8 - protein, 770 aa, Homo sapien
LDNPKCIDRSKPSEPIQPVNTWEISSLPSSTVTSETLPSSLSPSVEKYRYQDEDTPPQEHISPQITNEVIGPELVHVSEKNLSEIENVHGFVSHSHISPIKVNGTDADYEYEEITLERGNSGLGFSIAGGTDNPHIGDDSSIFITKIITGGAAAQDGRLRVNDCILRVNEVDVRDVTHSKAVEALKEAGSIVRLYVKRRKPVSEKIMEIKLIKGPKGLGFSIAGGVGNQHIPGDNSIYVTKIIEGGAAHKDGKLQIGDKLLAVNNVCLEEVTHEEAVTALKNTSDFVYLKVAKPTSMYMNDGYAPPDITNSSSQPVDNHVSPSSFLGQTPASPARYSPVSKAVLGDDEITREPRKVVLHRGSTGLGFNIVGGEDGEGIFISFILAGGPADLSGELRKGDRIISVNSVDLRAASHEQAAAALKNAGQAVTIVAQYRPEEYSRFEAKIHDLREQMMNSSISSGSGSLRTSQKRSLYVRALFDYDKTKDSGLPSQGLNFKFGDILHVINASDDEWWQARQVTPDGESDEVGVIPSKRRVEKKERARLKTVKFNSKTRDKGDSRGQEEYVLSYEPVNQQEVNYTRPVIILGPMKDRINDDLISEFPDKFGSCVPHTTRPKRDYEVDGRDYHFVTSREQMEKDIQEHKFIEAGQYNNHLYGTSVQSVREVAEKGKHCILDVSGNAIKRLQIAQLYPISIFIKPKSMENIMEMNKRLTEEQARKTFERAMKLEQEFTEHFTAIVQGDTLEDIYNQVKQIIEEQSGSYIWVPAKEKL

(19) United States
(12) Patent Application Publication
(10) Pub. No.: US 2014/0350078 A1
(43) Pub. Date: Nov. 27, 2014

(54) CONTROLLING REGULATORY T CELL FUNCTION

(71) Applicants: Michael Dustin, New York, NY (US); Alexandra Zanin-Zhorov, Staten Island, NY (US)

(72) Inventors: Michael Dustin, New York, NY (US); Alexandra Zanin-Zhorov, Staten Island, NY (US)

(73) Assignee: NEW YORK UNIVERSITY, New York, NY (US)

(21) Appl. No.: 14/344,891

(22) PCT Filed: Sep. 13, 2012
(86) PCT No.: PCT/US2012/055193

§ 371 Date: Jul. 9, 2014

Related U.S. Application Data
(60) Provisional application No. 61/534,272, filed on Sep. 13, 2011.

(51) Int. Cl. G01N 33/569; G01N 33/50; C12N 15/113

(52) U.S. Cl. CPC G01N 33/56966; C12N 15/113; G01N 33/505; C12N 2310/11; C12N 2320/30; G01N 2500/10
USPC 514/44 A; 435/7.92; 506/10

(57) ABSTRACT

The present invention relates to a method of identifying candidate compounds useful as chemotherapeutics or anti-infective compounds or anti-inflammatory drugs. This method involves providing a plurality of test compounds. The plurality of test compounds are incubated with human Regulatory T (Treg) cells expressing Disc-Large Homo log 1 (Dlgh1) or in which Dlgh1 is suppressed, where the Treg cells have an immunological synapse (IS). Test compounds which inhibit Dlgh1 expression, recruitment to the IS, and/or activity in the Treg cells are identified as candidate compounds potentially useful as chemotherapeutics or anti-infective compounds. Test compounds which enhance Dlgh1 recruitment to the IS and/or activity in the Treg cells are identified as candidate compounds potentially useful as anti-inflammatory drugs. The present invention also relates to methods of treating inflammatory conditions, cancers, and infectious diseases in a subject, as well as methods of inhibiting Treg cell activity.

This application claims the priority benefit of U.S. Provisional Patent Application Ser. No. 61/534,272, filed Sep. 13, 2011, which is hereby incorporated by reference in its entirety.

This invention was made with government support under grant numbers R37AI43542, PN2EY01696, R01AI41647, R56AI88553, 2RC2AR058986, and 2P01CA067493 awarded by the National Institutes of Health. The government has certain rights in this invention.

### FIELD OF THE INVENTION

This invention relates to controlling regulatory T cell function.

### BACKGROUND OF THE INVENTION

The immune system provides the human body with a means to recognize and defend itself against microorganisms, viruses, and substances recognized as foreign and potentially harmful. Classical immune responses are initiated when antigen-presenting cells present an antigen to CD4⁺ T helper (Th) lymphocytes resulting in T cell activation, proliferation, and differentiation of effector T lymphocytes. Following exposure to antigens, such as that which results from infection or the grafting of foreign tissue, naïve T cells differentiate into Th1, Th2, or Th17 cells with differing functions. Th1 cells produce interferon gamma (IFN-γ) and interleukin 2 (IL-2) and are important for beneficial responses to viruses and other intracellular pathogens. Deleterious side effects of Th1 cells include the rejection of foreign tissue grafts as well as many autoimmune diseases. Th2 cells produce cytokines such as interleukin-4 (IL-4), and are associated with beneficial responses to multicellular parasites such as intestinal worms. Deleterious Th2 responses include allergies and allergic inflammatory responses such as allergic rhinitis and asthma. Th2 cells may also contribute to the rejection of foreign grafts. Th17 cells are a relatively recently described population that produce IL-17 and IL-21 and mount beneficial responses against extracellular bacterial infections and the control of commensal bacteria in the gut. Th17 cells also play a critical early role in many autoimmune diseases. Th1, Th2, and Th17 cells plus naïve CD4⁺CD25⁻ T cells can be collectively defined as CD4⁺ effector T cells (Teff).

Regulatory T cells (Tregs) are a subset of CD4⁺ T cells that are central in maintaining a balance between immune tolerance to self-antigens and antitumor responses (Sakaguchi et al., “Regulatory T Cells and Immune Tolerance,” Cell 133:775-787 (2008)). Tregs are produced mainly in the thymus and require expression of the transcription factor Foxp3 for both development and function (Fontenot et al., “Foxp3 Programs the Development and Function of CD4+ CD25+ Regulatory T Cells,” Nat. Immunol. 4:330-336 (2003); Hori et al., “Control of Regulatory T Cell Development by the Transcription Factor Foxp3,” Science 299:1057-1061 (2003); Zheng et al., “Foxp3 in Control of the Regulatory T Cell Lineage,” Nat. Immunol. 8:457-462 (2007)). Foxp3 deficiency leads to defects in Treg function, which manifests as a multiorgan fatal inflammatory disease in mice (Sakaguchi, S., “Naturally Arising Foxp3-Expressing CD25+CD4+Regulatory T Cells in Immunological Tolerance to Self and Non-Self,” Nat. Immunol. 6:345-352 (2005); Shevach, E M., “Regulatory/Suppressor T Cells in Health and Disease,” Arthritis Rheum. 50:2721-2724 (2004)). In humans, Tregs isolated from the peripheral blood of patients with rheumatoid arthritis (RA) are defective in their ability to suppress effector T cell (Teff) function (Valencia et al., “TNF Downmodulates the Function of Human CD4+CD25hi T-Regulatory Cells,” Blood 108:253-261 (2006)). Although Treg activity is essential for prevention of autoimmunity, excessive Treg function may abrogate effective immune responses against tumor cells (Nishikawa et al., “Regulatory T Cells in Tumor Immunity,” Int. J. Cancer 127:759-767 (2010)). Indeed, down-regulation of Treg activity has been used as an effective tool to improve anticancer therapies (Grauer et al., “Elimination of Regulatory T Cells is Essential for an Effective Vaccination with Tumor Lysate-Pulsed Dendritic Cells in a Murine Glioma Model,” Int. J. Cancer 122:1794-1802 (2008); Zhou et al., “Depletion of Endogenous Tumor-Associated Regulatory T Cells Improves the Efficacy of Adoptive Cytotoxic T-Cell Immunotherapy in Murine Acute Myeloid Leukemia,” Blood 114:3793-3802 (2009)). Thus, the function of Tregs must be precisely controlled during immune responses to provide effective immunity without pathological anti-self reactivity.

Treg-suppressive function depends on T cell receptor (TCR) signaling (Shevach, E M. “Mechanisms of foxp3+ T Regulatory Cell-Mediated Suppression,” Immunity 30:636-645 (2009)), but the activating signals downstream of the TCRs that control Tregs are unknown and appear to be distinct from conventional T cells (Au-Yeung et al., “A Genetically Selective Inhibitor Demonstrates a Function for the Kinase Zap70 in Regulatory T Cells Independent of its Catalytic Activity,” Nat. Immunol. 11:1085-1092 (2010)). Scaffold proteins mediate linkages between receptors and signaling networks and thus have the potential to manipulate outcomes of receptor engagement (Rebeaud et al., “Dlgh1 and Carmal MAGUK Proteins Contribute to Signal Specificity Downstream of TCR Activation,” Trends Immunol. 28:196-200 (2007)). The membrane-associated guanylate kinases (MAGUKs) are scaffold proteins critical in development, growth control, and organization of neural synapses (Burack et al., “Signal Transduction: Hanging on a Scaffold,” Curr. Opin. Cell Biol. 12:211-216 (2000); Hanada et al., “Human Homologue of the Drosophila Discs Large Tumor Suppressor Binds to p561ck Tyrosine Kinase and Shaker Type Kv1.3 Potassium Channel in T Lymphocytes,” J. Biol. Chem. 272:26899-26904 (1997); Jordan et al., “Adaptors as Central Mediators of Signal Transduction in Immune Cells,” Nat. Immunol. 4:110-116 (2003)). MAGUKs are defined by a core module with a PDZ domain, an src homology 3 domain, and a catalytically inactive guanylate kinase domain (Anderson, J M., “Cell Signalling: MAGUK Magic,” Curr. Biol. 6:382-384 (1996)). Two MAGUKs have been implicated in the function of T cells: Carmal, which mediates negative feedback on Treg function downstream of the TCR; and Disc large homolog 1 (Dlgh1), which recruits p38 mitogen-activated protein kinase (p38) leading to Nuclear Factor of Activated T cells (NFATc1) activation in Teffs (Round et al., “Scaffold Protein Dlgh1 Coordinates Alternative p38 Kinase Activation, Directing T Cell Receptor Signals Toward NFAT but Not NF-kappaB Transcription Factors,” Nat. Immunol. 8:154-161 (2007)) and stabilizes the phosphatase and tensin homolog (PTEN) lipid phosphatase in oligodendrocytes (Adey et al., “Threonine Phosphorylation of the MMAC1/PTEN PDZ Binding Domain Both Inhibits and Stimulates PDZ Binding,” Cancer Res. 60:35-37 (2000); Cotter et al., “Dlg1-PTEN Interaction Regulates Myelin Thickness to Prevent Damaging Peripheral Nerve Overmyelination,” Science 328:1415-1418 (2010); Valiente et al., “Binding of PTEN to Specific PDZ Domains Contributes to PTEN Protein Stability and Phosphorylation by Microtubule-Associated Serine/Threonine Kinases,” J. Biol. Chem. 280:28936-28943 (2005)), thus suppressing Akt activation (Crellin et al., “Altered Activation of AKT is Required for the Suppressive Function of Human CD4+CD25+ T Regulatory Cells,” Blood 109:2014-2022 (2007); Wu et al., “FOXP3 Controls Regulatory T Cell Function Through Cooperation with NFAT,” Cell 126:375-387 (2006)).

TCR are released from T cell lines in culture and in individuals with disorders or diseases that involve T cell responses. These released receptors differ from the cellular membrane bound receptor and may be used therapeutically or diagnostically for certain T cell malignancies, and other diseases or disorders which elicit or involve T cell responses, including some infectious diseases, cancers, solid tumors, autoimmune diseases including rheumatoid arthritis, allergies etc.

One particular inflammatory disease, rheumatoid arthritis (RA), is a chronic, recurrent, inflammatory disease primarily involving joints, affecting 1-3% of North Americans. Severe RA patients tend to exhibit extra-articular manifestations including vasculitis, muscle atrophy, subcutaneous nodules, lymphadenopathy, splenomegaly, and leukopenia. Spontaneous remission may occur; other patients have brief episodes of acute arthritis with longer periods of low-grade activity; still others progress to severe deformity of joints. In some patients with RA, particularly those with long-standing disease, a constellation of symptoms called “Felty's syndrome” develops, in which the typical arthropathy is accompanied by splenomegaly and neutropenia. It is estimated that about 15% of RA patients (severe RA and Felty's syndrome) become completely incapacitated (Primer on the Rheumatic Diseases, 8th edition, Rodman, G. P. & Schumacher, H. R., Eds., Zvaifler, N. J., Assoc. Ed., Arthritis Foundations, Atlanta, Ga. (1983)). Despite advances in treatment, RA and other autoimmune diseases remain a serious health problem. Although rarely fatal, RA is a major cause of morbidity, lost time from work, lost productivity, and decreased quality of life. RA causes severe pain and loss of joint mobility and can make accomplishing even simple tasks difficult.

The antigenic stimulus initiating the immune response in RA and consequent inflammation is unknown. Certain human leukocyte antigen (HLA) types (DR4, Dw4, Dw14 and DR1) have an increased prevalence of RA, perhaps leading to a genetic susceptibility to an unidentified factor which initiates the disease process. The association with DR4 is highest for Felty's Disease and severe RA (Westedt et al., “Immunogenetic Heterogeneity of Rheumatoid Arthritis,” Annals of Rheumatic Diseases, 45:534-538 (1986)). Relationships between Epstein Barr virus and RA have been suggested. Synovial lymphocytes produce IgG that is recognized as foreign and stimulates a local immune response with production of anti-IgG-antibodies (rheumatoid factors). Immune complexes are formed by activation of the complement system which results in inflammation including activation of lysozyme and other enzymes. Helper T cell infiltration of the synovium and liberation of lymphokines such as IL6 lead to further accumulation of macrophages and slowly progressing joint destruction (erosions).

The approach to drug treatment in RA has been described as a pyramid (Primer on the Rheumatic Diseases, 8th edition, Rodman, G. P. & Schumacher, H. R., Eds., Zvaifler, N. J., Assoc. Ed., Arthritis Foundations, Atlanta, Ga. (1983)). First line agents include administration of non-steroidal anti-inflammatory drugs such as acetylsalicylic acid (aspirin), ibuprofen, naproxen, and other such agents. When these agents fail, gold salts, penicillamine, methotrexate, or antimalarials, known as conventional second line drugs, are considered. Finally, steroids or cytotoxics are tried in patients with serious active disease that is refractory to first and second line treatment. The most potent class of disease modifying anti-rheumatic drugs (DMARDS) are the anti-TNF therapies such as Embrel and Remicaid. These proteins are injected into the patient every couple of weeks for life and stop disease progression in about 30% of RA patients and generate significant slowing of disease progression in 70% of RA patients who have continued to progress rapidly with other therapies. Even with these recent improvements, there are still many patients whose disease is refractory to all current treatments.

Severe RA patients who were treated with total lymphoid irradiation or thoracic duct drainage experienced significant improvement of disease symptoms. These procedures are not suitable for routine application. Due to these encouraging findings, however, and to the demonstration of the presence of T cells in the synovial infiltrate, it is possible to design new immunotherapies to specifically eliminate T cells. Most of these new experimental immunotherapies are targeted toward all or the bulk of T cells, and thus may produce significant side effects. A better approach for selective immunotherapy may be to eliminate or regulate only the small proportion of T cells that are involved in RA.

Evidence has accumulated supporting a role for T cells in the pathogenesis of RA. The synovial tissue and surrounding synovial fluid of patients with RA are infiltrated with large numbers of cells. Activated Teffs can mediate tissue damage by a variety of mechanisms including the direct cytotoxicity of target cells expressing specific antigen in combination with the appropriate HLA restricting elements. The strong association of certain HLA products with RA has led researchers to implicate T cells in the autoimmune destruction of RA patient joints. In fact, HLA DR4, Dw4, and Dw14 gene products are among the major class II molecules that contribute significantly to disease susceptibility in RA patients (Todd et al., “A Molecular Basis for MHC Class II-Associated Autoimmunity,” Science 240:1003-1009 (1988)), and they are capable of restricting antigen recognition of CD4⁺ T cells, primarily. Other autoimmune diseases also show a high correlation between disease susceptibility and HLA expression.

In addition to inflammatory conditions like RA, there are a variety of other disorders such as cancer and infectious diseases, whose pathogenesis is closely related to the activity of Teffs. These disorders impact the lives of millions of people every day. Approximately 1.3 million people are diagnosed with cancer each year in the United States alone, and over 500,000 die. Treatment for most types of cancers include chemotherapy. Chemotherapy drugs are administered systemically and attack all cells of the body, particularly dividing cells, not just cancer cells. Thus, side effects from chemotherapy drugs are often severe. These include anemia, nausea, hair loss, and immune suppression, including neutropenia due to depletion of white blood cells. The side effects often limit the dose of chemotherapy agents that can be administered.

Similarly, infectious diseases are increasing in prevalence in the U.S. These include unhealthy conditions of the body or part thereof or the mind, which are caused by microorganisms such as bacteria, viruses, rickettsiae, chlamydiae, mycoplasmas, fungi, and protozoa. These microorganisms live on the skin, in the oral cavity, in the respiratory and gastrointestinal tracts, and in the genitalia, where they constitute normal flora. The interactions between the human hosts and pathogenic organisms are complex and dynamic and these interactions determine whether or not the microorganism remains apart from the human host, becomes part of the normal flora, or invades the host and causes disease. With the advent of new infectious diseases, such as new strains of influenza, there is a definite need for improved treatment thereof.

The present invention is directed to overcoming these and other deficiencies in the art.

### SUMMARY OF THE INVENTION

A first aspect of the present invention relates to a method of identifying candidate compounds useful as chemotherapeutics or anti-infective compounds or anti-inflammatory drugs. The method includes providing a plurality of test compounds. The plurality of test compounds are incubated with human Treg cells expressing Disc-Large Homolog 1 (Dlgh1) or in which Dlgh1 is suppressed, where the Treg cells have an immunological synapse. Test compounds which inhibit Dlgh1 expression, recruitment to the immunological synapse, and/or activity in the Treg cells are identified as candidate compounds potentially useful as chemotherapeutics or anti-infective compounds. Test compounds which enhance Dlgh1 recruitment to the immunological synapse, and/or activity in the Treg cells are identified as candidate compounds potentially useful as anti-inflammatory drugs.

Another aspect of the present invention relates to a method of treating an inflammatory condition in a subject. The method includes selecting a subject with an inflammatory condition and administering to the selected subject an agent which enhances Dlgh1 expression, recruitment to the immunological synapse, and/or activity under conditions effective to treat the inflammatory condition.

An additional aspect of the present invention relates to a method of treating cancer in a subject. The method includes selecting a subject with a cancer and administering to the selected subject an agent which inhibits Dlgh1 protein expression, recruitment to the immunological synapse, and/or activity in CD4⁺ CD25⁺ regulatory T cells under conditions effective to treat the cancer.

A further aspect of the present invention relates to a method of treating an infectious disease in a subject. The method includes selecting a subject with an infectious disease and administering to the selected subject an agent which inhibits Dlgh1 protein expression and/or activity under conditions effective to treat the infectious disease.

Another aspect of the present invention relates to a method of inhibiting regulatory T cell activity. The method includes administering to the regulatory T cell an agent that inhibits Dlgh1 protein expression under conditions effective to inhibit regulatory T cell activity.

The present invention is derived from the discovery that protein kinase C-theta (which is the product of the PRKCQ gene) acts as a negative regulator of Treg function down-stream of the TCR. Further, Human Disc Large-1 (which is the product of the SAP97 gene) is required for activation of Tregs. This protein is also referred to as Dlg-1, Dlgh1, synapse associated protein (Sap)-97. The effect of Dlgh1 likely represents a major pathway for Treg activation which was not previously known. The present invention reduces Dlgh1 protein through RNAi or other methods, which eliminates the activity of Treg. The local reduction of Treg activity is expected to promote anti-tumor immune responses and enhance vaccination efforts for cancer and infectious disease.

Because both NFAT activation and Akt suppression are important for activation of Treg-suppressive function, the present invention hypothesized that Dlgh1 could provide a missing link between the TCR and stimulation of Treg function. Indeed, the present invention found that Dlgh1 is strongly recruited to immunological synapse (1S) in Tregs compared with CD4⁺CD25⁻ T cells, which are enriched for Teff. Moreover, the decreased suppressive function of Tregs from RA patients or TNF-α-treated healthy Tregs correlates with diminished Dlgh1 recruitment to IS in those cells. Silencing of Dlgh1 gene expression abrogates the ability of human Tregs to suppress proliferation and cytokine secretion in CD4⁺CD25⁻ T cells, as well as down-regulates Foxp3 expression. Finally the present invention demonstrated that Dlgh1 is required for p38-mediated activation of NFAT and PTEN-mediated inhibition of AKT and NF-κB. Interestingly, Dlgh1 controls Treg function independently of the negative feedback pathway mediated by PKC-θ and related adaptor Carmal. The results of the present invention provide insight and fill gaps in the basic understanding of how TCR-induced signaling pathways are integrated and mediate positive feed-forward control of Treg function.

Foxp3⁺CD4⁺CD25^{high} Treg suppression of inflammation depends on TCR mediated NFATc1 activation and Akt inhibition. The role of the scaffold protein Dlgh1 in linking the T cell receptor to this unique signaling outcome was investigated in the present invention. Here, the Treg immunological synapse (IS) recruited 4-fold more Dlgh1 than conventional CD4⁺ T cells IS. Tregs isolated from patients with active rheumatoid arthritis or treated with tumor necrosis factor-α-treatment displayed reduced function, and had diminished Dlgh1 recruitment to the IS. Furthermore, Dlgh1 silencing abrogated Treg function, impaired NFATc1 activation, reduced PTEN levels, and increased Akt activation. Dlgh1 operates independently of the negative feedback pathway mediated by the related adapter protein Carmal and thus presents an array of unique targets to selectively manipulate Treg function.

### BRIEF DESCRIPTION OF THE DRAWINGS

FIGS. 1A-1C illustrate that Dlgh1 is strongly recruited to immunological synapse (IS) in Tregs. Freshly FACS-sorted (FIG. 1A) and MACS bead (FIG. 1B) purified human blood CD4⁺CD25^{hi} (Treg) and CD4⁺CD25⁻ T cells or expanded umbilical cord blood (UCB)-derived Treg and CD4⁺CD25⁻ T cells (FIG. 1C) were introduced into bilayers containing both anti-CD3 (5 μg/mL) and ICAM-1 at 250 molecules per mm² (FIGS. 1A and 1C) or anti-CD3 or ICAM-1 molecules alone (FIG. 1B), fixed at 8 minutes and permeabilized, stained with anti-Dlgh1 antibodies, and imaged by TIRFM. Shown are representative images. Dlgh1 staining was quantified by calculation of average fluorescence intensity in cells. Data are representative of three different experiments. P values were calculated by Mann-Whitney test.

FIGS. 2A-2B illustrate that Dlgh1 recruitment to IS correlates with Treg suppressive function. Freshly purified Tregs from healthy donors or RA patients were untreated (FIG. 2A) or TNF-α-treated (50 ng/mL for 24 hours) (FIG. 2B), introduced to bilayers with anti-CD3 and ICAM-1, fixed, and imaged by TIRFM. Shown are representative images. Dlgh1 staining was quantified by calculation of average fluorescence intensity in cells. Data are representative of seven (FIG. 2A) or three (FIG. 3B) different experiments. P values were calculated by Mann-Whitney test.

FIGS. 3A-3G show that Dlgh1 is required for Treg function. In FIG. 3A, freshly purified human Tregs were transfected with small interfering RNA (siRNA) targeting Dlgh1 or with control siRNA by AMAXA and plated in presence of IL-2 (300 IU/mL). After 48 hours, Dlgh1 expression was measured by Western blot analysis. In FIG. 3B, siRNA-transfected Tregs were mixed with CD4⁺CD25⁻ T cells at a 1:3 ratio and activated with anti-CD3/CD28 dynal beads. CD4⁺CD25⁻ T cell proliferation was determined after 96 hours by CFSE dilution. Representative experiment of three is shown. In FIG. 3C, the supernatants were analyzed for IFN-γ, IL-17, and IL-4 after 48 hours. In FIGS. 3D and 3E, Foxp3 expression was determined by flow cytometry 48 hours after Treg transfection (FIG. 3D), and the average of three different experiments is shown (FIG. 3E). In FIGS. 3F and 3G, some Tregs 48 hours after transfection were introduced to bilayers with anti-CD3 and ICAM-1, fixed, stained for PKC-θ, and imaged by confocal microscopy (FIG. 3F) or treated with PKC-θ inhibitor, C-20 (1 μM, 30 minutes), washed, and then mixed with untreated CD4⁻CD25⁻ T cells at ratio 1:3 (FIG. 3G). Average of three (FIGS. 3E-3G) or four (FIGS. 3A and 3C) different experiments are shown. P values were calculated by t test. *P<0.05.

FIGS. 4A-4F show that Dlgh1 controls Treg function via p38/NFAT and PTEN/Akt signaling pathways. In FIGS. 4A, 4B, 4D, and 4E, siRNA-transfected Tregs (48 hours after transfection) were activated (FIGS. 4A, 4B, and 4E) or not (FIG. 4D) by immobilized anti-CD3 antibodies (5 μg/mL) and lysed. In FIGS. 4A, 4D, and 4E, PTEN levels as well as p38 and AKT phosphorylation were determined by Western blot analysis. Numbers represent the intensity of specific bands divided by intensity of loading controls and multiplied by 100. Representative results of three independent experiments are shown. In FIG. 4B, NFATc1 activation (Left) and p50-specific binding to NF-κB consensus sequence (Right) were tested by ELISA. Average of three independent experiments is shown. In FIG. 4C, Dlgh1 coimmunoprecipitates with PTEN, but not with control IgG, in freshly purified human Tregs. Representative results of two independent experiments are shown. In FIG. 4F, Dlgh1 is required for activation of p38 and NFAT, which synergizes with Foxp3 and provides a positive feed-forward signal for Treg function. In addition, Dlgh1 interaction with PTEN inhibits AKT pathway that negatively regulates Treg function. P values were calculated by t test. *P<0.05.

FIGS. 5A-5D show the purification of human Tregs. In FIGS. 5A-5C, CD4⁺CD25^{high}CD127⁻ and CD4⁺CD25⁻ T cells were purified from healthy donors by sorting (˜90% of Foxp3⁺) (FIGS. 5A and 5B) or by positive selection by MACS (˜80% of CD4⁺CD25^{hi}) (FIGS. 5C). In FIG. 5D, Tregs and CD4⁺CD25⁻ T cells were stained for CD45RO and CD45 RA markers.

FIGS. 6A-6C show that Dlgh1 is enriched at IS in Tregs. In FIG. 6A, intracellular expression levels of Dlgh1 in Treg and CD4⁺CD25⁻ T cells were determined by FACS analysis in permeabilized cells, and one representative experiment of four is presented. In FIGS. 6B and 6C, Tregs and CD4⁺CD25⁻ T cells were introduced into bilayers containing anti-CD3 (5 μg/mL) and ICAM-1 at 250 molecules per mm² for 2, 8, and 20 minutes (FIG. 6B) or for 8 minutes with or without CD80 at 200 molecules per mm² (FIG. 6C), fixed and permeabilized, stained with anti-Dlgh1 antibodies, and imaged by TIRFM. Shown are representative images. Dlgh1 staining was quantified by calculation of average fluorescence intensity in cells. Data are representative of three different experiments. P values were calculated by Mann-Whitney test.

FIGS. 7A-7B show the clinical and demographic details of studied RA patients. M, male; F, female; AFI, average fluorescence intensity of Dlgh1 recruitment at IS (FIG. 7A). In FIG. 7B, intracellular expression levels of Dlgh1 were determined by FACS analysis in permeabilized CD4⁺ T cells purified from RA patients.

FIGS. 8A-8B show that Dlgh1 recruitment to IS is reduced after Dlgh1 silencing by specific siRNA in Treg. In FIG. 8A, CD4⁺CD25⁺ T cells were transfected with siRNA targeting Dlgh1 or with control siRNA. 24 hours later cells were introduced into bilayers containing anti-CD3 mAb (5 μg/mL) and ICAM-1 at 250 molecules per mm² for 8 minutes, fixed, permeabilized, stained for Dlgh1, and imaged by TIRF or by bright field microscopy (total Dlgh1). Images of randomly selected fields are shown. The average intensity of staining was measured. Data are representative of two different experiments. P values were calculated by Mann-Whitney test. In FIG. 8B, intracellular levels of Dlgh1 were determined by FACS, and representative experiment of two is shown.

FIGS. 9A-9D show that Dlgh1 is required for Treg function. In FIGS. 9A and 9B, freshly purified human Tregs were transfected with small interfering RNAs (siRNAs) targeting Dlgh1 (Dlgh1-1, Dlgh1-7, Dlgh1-8, Dlgh1-9, or mix of all) or with control siRNA by AMAXA and plated in presence of IL-2 (300 IU/mL). Dlgh1 (FIG. 9A) and Foxp3 (FIG. 9B) expression was determined by flow cytometry 48 hours after Treg transfection. In FIGS. 9C and 9D, siRNA-transfected Tregs were mixed with CD4⁺CD25⁻ T cells at 1:3 ratio and activated with anti-CD3/CD28 dynal beads (FIG. 9C) or on APCs with soluble anti-CD3 mAb (5 μg/mL) (FIG. 9D). The supernatants were analyzed for IFN-γ after 48 hours. Data are representative of three different experiments. P values were calculated by t test. **P<0.01.

FIGS. 10A-10B show that Dlgh1 silencing does not affect the localization of PKC-θ and Carma-1 in Treg. CD4⁺CD25⁺ T cells were transfected with siRNA targeting Dlgh1 or with control siRNA. 24 hours later cells were introduced into bilayers containing anti-CD3 mAb (5 μg/mL) and ICAM-1 at 250 molecules per mm² for 8 minutes, fixed, permeabilized, stained for PKC-θ (FIG. 10A) and Carma-1 (FIG. 10B), and imaged by TIRFM. Images of randomly selected fields are shown. The average intensity of staining was measured. Data are representative of two different experiments.

FIGS. 11A-11C show that Dlgh1 silencing has no effect on IFN-γ secretion and proliferation in CD4⁺CD25⁻ T cells. Freshly purified non-activated human CD4⁺CD25⁻ T cells were transfected with siRNA targeting Dlgh1 or with control siRNA by AMAXA and plated in the presence of IL-2 (300 IU/mL). In FIG. 11A, after 48 hours, Dlgh1 expression was measured by Western blot analysis and FACS. In FIGS. 11B and 11C, transfected cells were then activated on anti-CD3. The proliferation was measured by CFSE dilution by FACS after 96 hours (FIG. 11B) and IFN-γ secretion by ELISA after 48 hours (FIG. 11C). *P<0.01 vs control. Data are representative of three different experiments. *P<0.05 vs control.

FIG. 12 shows that p38 kinase is required for Treg function. Freshly purified human Tregs were treated with p38 kinase inhibitor SB203580 or with PKC-θ inhibitor C-20 at 1 μM for 30 minutes, washed three times, and mixed with CD4⁺CD25⁻ T cells at 1:3 and plated on immobilized anti-CD3 mAb. The supernatants were analyzed for IFN-γ secretion after 48 hours. The means±SD of three different experiments are shown. P values were calculated by t test. *P<0.05.

FIGS. 13A-13C show that Dlgh1 silencing down-regulates PTEN expression and up-regulates TCR-induced phosphorylation of AKT in CD4⁺CD25⁻ T cells. SiRNA transfected CD4⁺CD25⁻ T cells (48 hours after transfection) were activated (FIGS. 13B and 13C) or not (FIG. 13A) by immobilized anti-CD3 antibodies (5 μg/mL) and lysed. PTEN levels (FIG. 13A) as well as AKT (FIG. 13B) and p38 (FIG. 13C) phosphorylation were determined by Western blot analysis. Numbers represent the intensity of specific bands divided by intensity of loading controls and multiplied by 100. Representative results of three independent experiments are shown.

### DETAILED DESCRIPTION OF THE INVENTION

A first aspect of the present invention relates to a method of identifying candidate compounds useful as chemotherapeutics or anti-infective compounds or anti-inflammatory drugs. The method includes providing a plurality of test compounds. The plurality of test compounds are incubated with human Regulatory T (Treg) cells expressing Disc-Large Homolog 1 (Dlgh1) or in which Dlgh1 is suppressed, where the Treg cells have an immunological synapse. Test compounds which inhibit Dlgh1 expression, recruitment to the immunological synapse, and/or activity in the Treg cells are identified as candidate compounds potentially useful as chemotherapeutics or anti-infective compounds. Test compounds which enhance Dlgh1 recruitment to the immunological synapse, and/or activity in the Treg cells are identified as candidate compounds potentially useful as anti-inflammatory drugs.

As used herein, the term chemotherapeutic compound refers to a synthetic, biological, or semi-synthetic compound that is not an enzyme and that kills cancer cells or inhibits the growth of cancer cells while having less effect on non-cancerous cells.

Suitable chemotherapeutic compounds that can be used in accordance with the present invention include, but are not limited to, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorabicin, bleomycin, plicomycin, mitomycin, etoposide (VP 16), tamoxifen, raloxifene, estrogen receptor binding agents, taxol, gemcitabien, navelbine, farnesyl-protein transferase inhibitors, transplatinum, 5-fluorouracil, vincristine, vinblastine and methotrexate, Temazolomide (an aqueous form of DTIC), or any analog or derivative variant of the foregoing.

The chemotherapeutic compounds of the present aspect can be administered as part of a combination therapy in conjunction with another cancer treatment, depending upon the nature of the cancer that is being treated. Such additional cancer treatments include, but are not limited to, radiotherapy, immunotherapy, and surgery. Types of radiotherapy that are contemplated by the present invention include, but are not limited to, the use of γ-rays, X-rays, and/or the directed delivery of radioisotopes to tumor cells. Other forms of DNA damaging factors are also contemplated such as microwaves and UV-irradiation. Immunotherapy according to the present invention, generally rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionucleotide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. Surgery according to the present invention can include, but is not limited to, resection in which all or part of cancerous or other relevant tissue is physically removed, excised, and/or destroyed. Tumor resection includes physical removal of at least part of a tumor. In addition to tumor resection, treatment by surgery includes laser surgery, cryosurgery, electrosurgery, and microscopically controlled surgery (Mohs' surgery), laparascopic surgery and harmonic scalpel surgery. For additional examples of combination therapies for cancer treatment according to the present invention, see WO 2007/139556 to Meyer et al., which is hereby incorporated by reference in its entirety.

Similarly, as used herein, the term anti-infective compound is intended to include compounds that reduce the virulence of a pathogen to a host organism and compounds that inhibit the growth of or kill a pathogen in the presence and/or in the absence of a host organism like antibiotic compounds.

Suitable anti-infective compounds of the present invention can include any synthetic or semi-synthetic compound. Such compounds include inorganic as well as organic chemical compounds. The compounds may be as well naturally occurring compounds. Naturally occurring compounds may include, e.g., saccharides, lipids, peptides, proteins, nucleic acids, or combinations thereof, e.g., aminoglycosides, glycolipids, lipopolysaccharides, or macrolides. The precise source of the compound is not critical to the method of the present invention. The compound might be derived from e.g., synthetic compounds libraries which are commercially available, e.g., Sigma-Aldrich (Milwaukee, Wis.), or libraries of natural occurring compounds in the form of bacterial, fungal, plant, and animal extracts such as those available from Xenova (Slough, UK) might be chemically synthesized or produced by recombinant technologies according to methods known to the person skilled in the art. The synthetic (or semi-synthetic), natural occurring compounds or bacterial, fungal, plant, and animal extracts might be modified using standard chemical, physical, or biochemical methods known in the art. One compound or multiple compounds may be used in the present invention. In one embodiment, the candidate compounds that are potentially useful as anti-infective compounds are identified.

The term anti-inflammatory drug as used herein refers to a synthetic, biological, or semi-synthetic compound that is not an enzyme and that reduces or eliminates inflammation or inhibits inflammation. In one embodiment, candidate compounds that are potentially useful as anti-inflammatory drugs are identified.

As used herein, the term Dlgh1, also referred to as discs, large homolog 1 (Drosophila), DKFZp761P0818, DLGH1, SAP-97, SAP97, dJ1061C18.1.1, hDlg, and hdlg refers to the human homolog of the Drosophila lethal (1) discs large-1 (dig) tumor suppressor (see, for example, Round et al., “Scaffold Protein Dlgh1 Coordinates Alternative p38 Kinase Activation, Directing T Cell Receptor Signals Toward NFAT but not NF-kappaB Transcription Factors,” Nat. Immunol. 8(2):154-61 (2007), which is hereby incorporated by reference in its entirety). Dlgh1 is a founding member of the membrane-associated guanylate kinase family of proteins containing PostSynaptic Density-95/Discs large/Zona Occludens-1 domains and is an ortholog of the Drosophila tumor suppressor gene Discs large. In the mammalian embryo, Dlgh1 is essential for normal urogenital morphogenesis and the development of skeletal and epithelial structures (Stephenson et al., “DLGH1 Is a Negative Regulator of T-Lymphocyte Proliferation,” Mol. Cell. Biol. 27(21):7574-7581 (2007), which is hereby incorporated by reference in its entirety).

The amino acid sequence of human Dlgh1 is known and can be found in, for example, GenBank accession number gi:4758162. The nucleotide sequence of human Dlgh1 be found in, for example, GenBank accession number gi:4758161. The nucleotide and amino acid sequence of mouse Dlgh1 may be found in, for example, GenBank accession number gi:40254641. The amino acid sequence of Dlgh1 proteins from various isoforms of Dlgh1 that are suitable for the present invention are shown in Table 1 below (i.e., SEQ ID NOs:1-7). SEQ ID NO:8 of Table 1 is a Dlgh1 consensus sequence demonstrating the high level of sequence identity across Dlgh1 proteins.

TABLE 1
Disks Large Homolog 1 Homo sapiens Sequence Alignment
Dlgh1 Isoform

| | | | |
|---|---|---|---|
| spQ12959-6 | LDNPKCIDRSKPSEPIQPVNTWEISSLPSSTVTSETLPSSLSPSVEKYRYQDEDTPPQEH | 60 | SEQ ID NO: 1 |
| spQ12959-7 | LDNPKCIDRSKPSEPIQPVNTWEISSLPSSTVTSETLPSSLSPSVEKYRYQDEDTPPQEH | 60 | SEQ ID NO: 2 |
| spQ12959 | LDNPKCIDRSKPSEPIQPVNTWEISSLPSSTVTSETLPSSLSPSVEKYRYQDEDTPPQEH | 60 | SEQ ID NO: 3 |
| spQ12959-5 | LDNPKCIDRSKPSEPIQPVNTWEISSLPSSTVTSETLPSSLSPSVEKYRYQDEDTPPQEH | 60 | SEQ ID NO: 4 |
| spQ12959-3 | LDNPKCIDRSKPSEPIQPVNTWEISSLPSSTVTSETLPSSLSPSVEKYRYQDEDTPPQEH | 60 | SEQ ID NO: 5 |
| spQ12959-2 | LDNPKCIDRSKPSEPIQPVNTWEISSLPSSTVTSETLPSSLSPSVEKYRYQDEDTPPQEH | 60 | SEQ ID NO: 6 |
| spQ12959-4 | LDNPKCIDRSKPSEPIQPVNTWEISSLPSSTVTSETLPSSLSPSVEKYRYQDEDTPPQEH | 60 | SEQ ID NO: 7 |
| | ************************************************************ | | |
| Consensus | LDNPKCIDRSKPSEPIQPVNTWEISSLPSSTVTSETLPSSLSPSVEKYRYQDEDTPPQEH | | SEQ ID NO: 8 |
| Sequence | | | |
| spQ12959-6 | ISPQITNEVIGPELVHVSEKNLSEIENVHGFVSHSHISPIKPTEAVLPSPPTVPVIPVLP | 120 | |
| spQ12959-7 | ISPQITNEVIGPELVHVSEKNLSEIENVHGFVSHSHISPIKPTEAVLPSPPTVPVIPVLP | 120 | |
| spQ12959 | ISPQITNEVIGPELVHVSEKNLSEIENVHGFVSHSHISPIKPTEAVLPSPPTVPVIPVLP | 120 | |
| spQ12959-5 | ISPQITNEVIGPELVHVSEKNLSEIENVHGFVSHSHISPIK------------------- | 101 | |
| spQ12959-3 | ISPQITNEVIGPELVHVSEKNLSEIENVHGFVSHSHISPIK------------------- | 101 | |
| spQ12959-2 | ISPQITNEVIGPELVHVSEKNLSEIENVHGFVSHSHISPIKPTEAVLPSPPTVPVIPVLP | 120 | |
| spQ12959-4 | ISPQITNEVIGPELVHVSEKNLSEIENVHGFVSHSHISPIK------------------- | 101 | |
| | ***************************************** | | |
| Consensus | ISPQITNEVIGPELVHVSEKNLSEIENVHGFVSHSHISPIK------------------- | | |
| Sequence | | | |
| spQ12959-6 | VPAENTVILPTIPQANPPPVLVNTDSLETPTYVNGTDADYEYEEITLERGNSGLGFSIAG | 180 | |
| spQ12959-7 | VPAENTVILPTIPQANPPPVLVNTDSLETPTYVNGTDADYEYEEITLERGNSGLGFSIAG | 180 | |
| spQ12959 | VPAENTVILPTIPQANPPPVLVNTDSLETPTYVNGTDADYEYEEITLERGNSGLGFSIAG | 180 | |
| spQ12959-5 | --------------------------------VNGTDADYEYEEITLERGNSGLGFSIAG | 129 | |
| spQ12959-3 | --------------ANPPPVLVNTDSLETPTYVNGTDADYEYEEITLERGNSGLGFSIAG | 147 | |
| spQ12959-2 | VPAENTVILPTIPQANPPPVLVNTDSLETPTYVNGTDADYEYEEITLERGNSGLGFSIAG | 180 | |
| spQ12959-4 | --------------ANPPPVLVNTDSLETPTYVNGTDADYEYEEITLERGNSGLGFSIAG | 147 | |
| | **************************** | | |
| Consensus | --------------------------------VNGTDADYEYEEITLERGNSGLGFSIAG | | |
| Sequence | | | |
| spQ12959-6 | GTDNPHIGDDSSIFITKIITGGAAAQDGRLRVNDCILRVNEVDVRDVTHSKAVEALKEAG | 240 | |
| spQ12959-7 | GTDNPHIGDDSSIFITKIITGGAAAQDGRLRVNDCILRVNEVDVRDVTHSKAVEALKEAG | 240 | |
| spQ12959 | GTDNPHIGDDSSIFITKIITGGAAAQDGRLRVNDCILRVNEVDVRDVTHSKAVEALKEAG | 240 | |
| spQ12959-5 | GTDNPHIGDDSSIFITKIITGGAAAQDGRLRVNDCILRVNEVDVRDVTHSKAVEALKEAG | 189 | |
| spQ12959-3 | GTDNPHIGDDSSIFITKIITGGAAAQDGRLRVNDCILRVNEVDVRDVTHSKAVEALKEAG | 207 | |
| spQ12959-2 | GTDNPHIGDDSSIFITKIITGGAAAQDGRLRVNDCILRVNEVDVRDVTHSKAVEALKEAG | 240 | |
| spQ12959-4 | GTDNPHIGDDSSIFITKIITGGAAAQDGRLRVNDCILRVNEVDVRDVTHSKAVEALKEAG | 207 | |
| | ************************************************************ | | |
| Consensus | GTDNPHIGDDSSIFITKIITGGAAAQDGRLRVNDCILRVNEVDVRDVTHSKAVEALKEAG | | |
| Sequence | | | |
| spQ12959-6 | SIVRLYVKRRKPVSEKIMEIKLIKGPKGLGFSIAGGVGNQHIPGDNSIYVTKIIEGGAAH | 300 | |
| spQ12959-7 | SIVRLYVKRRKPVSEKIMEIKLIKGPKGLGFSIAGGVGNQHIPGDNSIYVTKIIEGGAAH | 300 | |
| spQ12959 | SIVRLYVKRRKPVSEKIMEIKLIKGPKGLGFSIAGGVGNQHIPGDNSIYVTKIIEGGAAH | 300 | |
| spQ12959-5 | SIVRLYVKRRKPVSEKIMEIKLIKGPKGLGFSIAGGVGNQHIPGDNSIYVTKIIEGGAAH | 249 | |
| spQ12959-3 | SIVRLYVKRRKPVSEKIMEIKLIKGPKGLGFSIAGGVGNQHIPGDNSIYVTKIIEGGAAH | 267 | |
| spQ12959-2 | SIVRLYVKRRKPVSEKIMEIKLIKGPKGLGFSIAGGVGNQHIPGDNSIYVTKIIEGGAAH | 300 | |
| spQ12959-4 | SIVRLYVKRRKPVSEKIMEIKLIKGPKGLGFSIAGGVGNQHIPGDNSIYVTKIIEGGAAH | 267 | |
| | ************************************************************ | | |
| Consensus | SIVRLYVKRRKPVSEKIMEIKLIKGPKGLGFSIAGGVGNQHIPGDNSIYVTKIIEGGAAH | | |
| Sequence | | | |
| spQ12959-6 | KDGKLQIGDKLLAVNNVCLEEVTHEEAVTALKNTSDFVYLKVAKPTSMYMNDGYAPPDIT | 360 | |
| spQ12959-7 | KDGKLQIGDKLLAVNNVCLEEVTHEEAVTALKNTSDFVYLKVAKPTSMYMNDGYAPPDIT | 360 | |
| spQ12959 | KDGKLQIGDKLLAVNNVCLEEVTHEEAVTALKNTSDFVYLKVAKPTSMYMNDGYAPPDIT | 360 | |
| spQ12959-5 | KDGKLQIGDKLLAVNNVCLEEVTHEEAVTALKNTSDFVYLKVAKPTSMYMNDGYAPPDIT | 309 | |
| spQ12959-3 | KDGKLQIGDKLLAVNNVCLEEVTHEEAVTALKNTSDFVYLKVAKPTSMYMNDGYAPPDIT | 327 | |
| spQ12959-2 | KDGKLQIGDKLLAVNNVCLEEVTHEEAVTALKNTSDFVYLKVAKPTSMYMNDGYAPPDIT | 360 | |
| spQ12959-4 | KDGKLQIGDKLLAVNNVCLEEVTHEEAVTALKNTSDFVYLKVAKPTSMYMNDGYAPPDIT | 327 | |
| | ************************************************************ | | |
| Consensus | KDGKLQIGDKLLAVNNVCLEEVTHEEAVTALKNTSDFVYLKVAKPTSMYMNDGYAPPDIT | | |
| Sequence | | | |
| spQ12959-6 | NSSSQPVDNHVSPSSFLGQTPASPARYSPVSKAVLGDDEITREPRKVVLHRGSTGLGFNI | 420 | |
| spQ12959-7 | NSSSQPVDNHVSPSSFLGQTPASPARYSPVSKAVLGDDEITREPRKVVLHRGSTGLGFNI | 420 | |
| spQ12959 | NSSSQPVDNHVSPSSFLGQTPASPARYSPVSKAVLGDDEITREPRKVVLHRGSTGLGFNI | 420 | |
| spQ12959-5 | NSSSQPVDNHVSPSSFLGQTPASPARYSPVSKAVLGDDEITREPRKVVLHRGSTGLGFNI | 369 | |
| spQ12959-3 | NSSSQPVDNHVSPSSFLGQTPASPARYSPVSKAVLGDDEITREPRKVVLHRGSTGLGFNI | 387 | |
| spQ12959-2 | NSSSQPVDNHVSPSSFLGQTPASPARYSPVSKAVLGDDEITREPRKVVLHRGSTGLGFNI | 420 | |
| spQ12959-4 | NSSSQPVDNHVSPSSFLGQTPASPARYSPVSKAVLGDDEITREPRKVVLHRGSTGLGFNI | 387 | |
| | ************************************************************ | | |
| Consensus | NSSSQPVDNHVSPSSFLGQTPASPARYSPVSKAVLGDDEITREPRKVVLHRGSTGLGFNI | | |
| Sequence | | | |
| spQ12959-6 | VGGEDGEGIFISFILAGGPADLSGELRKGDRIISVNSVDLRAASHEQAAAALKNAGQAVT | 480 | |
| spQ12959-7 | VGGEDGEGIFISFILAGGPADLSGELRKGDRIISVNSVDLRAASHEQAAAALKNAGQAVT | 480 | |
| spQ12959 | VGGEDGEGIFISFILAGGPADLSGELRKGDRIISVNSVDLRAASHEQAAAALKNAGQAVT | 480 | |
| spQ12959-5 | VGGEDGEGIFISFILAGGPADLSGELRKGDRIISVNSVDLRAASHEQAAAALKNAGQAVT | 429 | |
| spQ12959-3 | VGGEDGEGIFISFILAGGPADLSGELRKGDRIISVNSVDLRAASHEQAAAALKNAGQAVT | 447 | |
| spQ12959-2 | VGGEDGEGIFISFILAGGPADLSGELRKGDRIISVNSVDLRAASHEQAAAALKNAGQAVT | 480 | |
| spQ12959-4 | VGGEDGEGIFISFILAGGPADLSGELRKGDRIISVNSVDLRAASHEQAAAALKNAGQAVT | 447 | |
| | ************************************************************ | | |
| Consensus | VGGEDGEGIFISFILAGGPADLSGELRKGDRIISVNSVDLRAASHEQAAAALKNAGQAVT | | |
| Sequence | | | |
| spQ12959-6 | IVAQYRPEEYSRFEAKIHDLREQMMNSSISSGSGSLRTSQKRSLYVRALFDYDKTKDSGL | 540 | |
| spQ12959-7 | IVAQYRPEEYSRFEAKIHDLREQMMNSSISSGSGSLRTSQKRSLYVRALFDYDKTKDSGL | 540 | |
| spQ12959 | IVAQYRPEEYSRFEAKIHDLREQMMNSSISSGSGSLRTSQKRSLYVRALFDYDKTKDSGL | 540 | |
| spQ12959-5 | IVAQYRPEEYSRFEAKIHDLREQMMNSSISSGSGSLRTSQKRSLYVRALFDYDKTKDSGL | 489 | |
| spQ12959-3 | IVAQYRPEEYSRFEAKIHDLREQMMNSSISSGSGSLRTSQKRSLYVRALFDYDKTKDSGL | 507 | |
| spQ12959-2 | IVAQYRPEEYSRFEAKIHDLREQMMNSSISSGSGSLRTSQKRSLYVRALFDYDKTKDSGL | 540 | |
| spQ12959-4 | IVAQYRPEEYSRFEAKIHDLREQMMNSSISSGSGSLRTSQKRSLYVRALFDYDKTKDSGL | 507 | |
| | ************************************************************ | | |
| Consensus | IVAQYRPEEYSRFEAKIHDLREQMMNSSISSGSGSLRTSQKRSLYVRALFDYDKTKDSGL | | |
| Sequence | | | |
| spQ12959-6 | PSQGLNFKFGDILHVINASDDEWWQARQVTPDGESDEVGVIPSKRRVEKKERARLKTVKF | 600 | |
| spQ12959-7 | PSQGLNFKFGDILHVINASDDEWWQARQVTPDGESDEVGVIPSKRRVEKKERARLKTVKF | 600 | |
| spQ12959 | PSQGLNFKFGDILHVINASDDEWWQARQVTPDGESDEVGVIPSKRRVEKKERARLKTVKF | 600 | |
| spQ12959-5 | PSQGLNFKFGDILHVINASDDEWWQARQVTPDGESDEVGVIPSKRRVEKKERARLKTVKF | 549 | |
| spQ12959-3 | PSQGLNFKFGDILHVINASDDEWWQARQVTPDGESDEVGVIPSKRRVEKKERARLKTVKF | 567 | |
| spQ12959-2 | PSQGLNFKFGDILHVINASDDEWWQARQVTPDGESDEVGVIPSKRRVEKKERARLKTVKF | 600 | |
| spQ12959-4 | PSQGLNFKFGDILHVINASDDEWWQARQVTPDGESDEVGVIPSKRRVEKKERARLKTVKF | 567 | |
| | ************************************************************ | | |
| Consensus | PSQGLNFKFGDILHVINASDDEWWQARQVTPDGESDEVGVIPSKRRVEKKERARLKTVKF | | |
| Sequence | | | |
| spQ12959-6 | NSKTRDKGEIPDDMG----------------------SKGLK------------------ | 620 | |
| spQ12959-7 | NSKTRDKGEIPDDMG----------------------SKGLKHVTSNASDSESSYLILIT | 638 | |
| spQ12959 | NSKTRDKGEIPDDMG----------------------SKGLKHVTSNASDSESS------ | 632 | |
| spQ12959-5 | NSKTRDKGEIPDDMG----------------------SKGLKHVTSNASDSESS------ | 581 | |
| spQ12959-3 | NSKTRDKGEIPDDMG----------------------SKGLKHVTSNASDSESS------ | 599 | |
| spQ12959-2 | NSKTRDKGQSFNDKRKKNLFSRKFPFYKNKDQSEQETSDADQHVTSNASDSESS------ | 654 | |
| spQ12959-4 | NSKTRDKGQSFNDKRKKNLFSRKFPFYKNKDQSEQETSDADQHVTSNASDSESS------ | 621 | |
| | ********: :* *.. : | | |
| Consensus | NSKTRDKG----D------------------------S---------------------- | | |
| Sequence | | | |
| spQ12959-6 | --------RGQEEYVLSYEPVNQQEVNYTRPVIILGPMKDRINDDLISEFPDKFGSCVPH | 672 | |
| spQ12959-7 | DEYGCSKGRGQEEYVLSYEPVNQQEVNYTRPVIILGPMKDRINDDLISEFPDKFGSCVPH | 698 | |
| spQ12959 | -------YRGQEEYVLSYEPVNQQEVNYTRPVIILGPMKDRINDDLISEFPDKFGSCVPH | 685 | |
| spQ12959-5 | -------YRGQEEYVLSYEPVNQQEVNYTRPVIILGPMKDRINDDLISEFPDKFGSCVPH | 634 | |
| spQ12959-3 | -------YRGQEEYVLSYEPVNQQEVNYTRPVIILGPMKDRINDDLISEFPDKFGSCVPH | 652 | |
| spQ12959-2 | -------YRGQEEYVLSYEPVNQQEVNYTRPVIILGPMKDRINDDLISEFPDKFGSCVPH | 707 | |
| spQ12959-4 | -------YRGQEEYVLSYEPVNQQEVNYTRPVIILGPMKDRINDDLISEFPDKFGSCVPH | 674 | |
| | **************************************************** | | |
| Consensus | --------RGQEEYVLSYEPVNQQEVNYTRPVIILGPMKDRINDDLISEFPDKFGSCVPH | | |
| Sequence | | | |
| spQ12959-6 | TTRPKRDYEVDGRDYHFVTSREQMEKDIQEHKFIEAGQYNNHLYGTSVQSVREVAEKGKH | 732 | |
| spQ12959-7 | TTRPKRDYEVDGRDYHFVTSREQMEKDIQEHKFIEAGQYNNHLYGTSVQSVREVAEKGKH | 758 | |
| spQ12959 | TTRPKRDYEVDGRDYHFVTSREQMEKDIQEHKFIEAGQYNNHLYGTSVQSVREVAEKGKH | 745 | |
| spQ12959-5 | TTRPKRDYEVDGRDYHFVTSREQMEKDIQEHKFIEAGQYNNHLYGTSVQSVREVAEKGKH | 694 | |
| spQ12959-3 | TTRPKRDYEVDGRDYHFVTSREQMEKDIQEHKFIEAGQYNNHLYGTSVQSVREVAEKGKH | 712 | |
| spQ12959-2 | TTRPKRDYEVDGRDYHFVTSREQMEKDIQEHKFIEAGQYNNHLYGTSVQSVREVAEKGKH | 767 | |
| spQ12959-4 | TTRPKRDYEVDGRDYHFVTSREQMEKDIQEHKFIEAGQYNNHLYGTSVQSVREVAEKGKH | 734 | |
| | ************************************************************ | | |
| Consensus | TTRPKRDYEVDGRDYHFVTSREQMEKDIQEHKFIEAGQYNNHLYGTSVQSVREVAEKGKH | | |
| Sequence | | | |
| spQ12959-6 | CILDVSGNAIKRLQIAQLYPISIFIKPKSMENIMEMNKRLTEEQARKTFERAMKLEQEFT | 792 | |
| spQ12959-7 | CILDVSGNAIKRLQIAQLYPISIFIKPKSMENIMEMNKRLTEEQARKTFERAMKLEQEFT | 818 | |
| spQ12959 | CILDVSGNAIKRLQIAQLYPISIFIKPKSMENIMEMNKRLTEEQARKTFERAMKLEQEFT | 805 | |
| spQ12959-5 | CILDVSGNAIKRLQIAQLYPISIFIKPKSMENIMEMNKRLTEEQARKTFERAMKLEQEFT | 754 | |
| spQ12959-3 | CILDVSGNAIKRLQIAQLYPISIFIKPKSMENIMEMNKRLTEEQARKTFERAMKLEQEFT | 772 | |
| spQ12959-2 | CILDVSGNAIKRLQIAQLYPISIFIKPKSMENIMEMNKRLTEEQARKTFERAMKLEQEFT | 827 | |
| spQ12959-4 | CILDVSGNAIKRLQIAQLYPISIFIKPKSMENIMEMNKRLTEEQARKTFERAMKLEQEFT | 794 | |
| | ************************************************************ | | |
| Consensus | CILDVSGNAIKRLQIAQLYPISIFIKPKSMENIMEMNKRLTEEQARKTFERAMKLEQEFT | | |
| Sequence | | | |
| spQ12959-6 | EHFTAIVQGDTLEDIYNQVKQIIEEQSGSYIWVPAKEKL | 831 | |
| spQ12959-7 | EHFTAIVQGDTLEDIYNQVKQIIEEQSGSYIWVPAKEKL | 857 | |
| spQ12959 | EHFTAIVQGDTLEDIYNQVKQIIEEQSGSYIWVPAKEKL | 844 | |
| spQ12959-5 | EHFTAIVQGDTLEDIYNQVKQIIEEQSGSYIWVPAKEKL | 793 | |
| spQ12959-3 | EHFTAIVQGDTLEDIYNQVKQIIEEQSGSYIWVPAKEKL | 811 | |
| spQ12959-2 | EHFTAIVQGDTLEDIYNQVKQIIEEQSGSYIWVPAKEKL | 866 | |
| spQ12959-4 | EHFTAIVQGDTLEDIYNQVKQIIEEQSGSYIWVPAKEKL | 833 | |
| | *************************************** | | |
| Consensus | EHFTAIVQGDTLEDIYNQVKQIIEEQSGSYIWVPAKEKL | | |
| Sequence | | | |

As used herein, the term immunological synapse (1S) refers to the interface between an antigen-presenting cell and a Treg lymphocyte cell. T cell recognition of antigen presenting cells (APCs) results in the formation of this specialized interface, the IS (Bromley et al., “The Immunological Synapse,” Annu. Rev. Immunol. 19:375-396 (2001); van der Mei-we, “Formation and Function of the Immunological Synapse,” Curr. Opin. Immunol. 14:293-298 (2002); Delon et al., “Information Transfer at the Immunological Synapse,” Curr. Biol. 10:R923-R933 (2000), which are hereby incorporated by reference in their entirety). The interaction of T cell antigen receptors with major histocompatibility complex molecule-peptide complexes in the nanometer scale gap at the immunological synapse initiates the adaptive immune response (Bromley et al., “The Immunological Synapse,” Annu. Rev. Immunol. 19:375-396 (2001), which is hereby incorporated by reference in its entirety). The IS is a specialized cell-cell junction between T cell and antigen-presenting cell surfaces (Lee et al., “The Immunological Synapse Balances T Cell Receptor Signaling and Degradation,” Science 302(5648): 1218-1222 (2003), which is hereby incorporated by reference in its entirety). It is characterized by a central cluster of antigen receptors, a ring of integrin family adhesion molecules, and temporal stability over hours (Lee et al., “The Immunological Synapse Balances T Cell Receptor Signaling and Degradation,” Science 302(5648): 1218-1222 (2003), which is hereby incorporated by reference in its entirety).

The IS acts as a type of adaptive controller that both boosts T cell receptor triggering and attenuates strong signals. It also functions to integrate and/or stabilize TCR-generated signaling pathways and to promote the restricted delivery of secretory products, such as cytokines, to the target cell.

Engagement of the TCR complex initiates signal transduction pathways involving protein and lipid kinases, phosphatases, and adapter proteins. Signals emanating from the TCR are thought to be modulated by the recruitment of transmembrane and cytosolic adaptor proteins, including PostSynaptic Density-95/Discs large/Zona Occludens 1 (“PDZ”) domain-containing scaffolds of the MAGUK family. As used herein, PDZ domains can bind to proteins via several mechanisms, the most common of which is through binding of conserved carboxyl-terminal sequences of proteins (Giallourakis et al., “A Molecular-Properties-Based Approach to Understanding PDZ Domain Proteins and PDZ Ligands,” Genome Res. 16:1056-1072 (2006) and Sheng et al., “PDZ Domains and the Organization of Supramolecular Complexes,” Annu. Rev. Neurosci. 24:1-29 (2001), which are hereby incorporated by reference in their entirety). Dlgh1 is critical for regulation of T cell proliferative responses. The role of a MAGUK protein in lymphocytes indicates that Dlgh1 functions in the negative regulation of T-lymphocyte proliferation (Stephenson et al., “DLGH1 Is a Negative Regulator of T-Lymphocyte Proliferation,” Mol. Cell. Biol. 27(21):7574-7581 (2007), which is hereby incorporated by reference in its entirety).

In one embodiment, the test compound or agent may enhance Dlgh1 protein expression. In another embodiment, the test compound or agent may reduce or silence Dlgh1 protein expression. As used herein in the present methods, Dlgh1 may be enhanced or suppressed in a number of ways. For example, Dlgh1 may be enhanced or suppressed by an interfering RNA (“RNAi”) molecule, which when introduced into a targeted cell enhances or inhibits Dlgh1 expression and, thus phosphorylation thereof. In certain embodiments, the RNAi agent is siRNA, shRNA, miRNA, or an antisense RNA molecule that enhances or disrupts stability of Dlgh1. The most preferable method of enhancement or suppression in the present methods is by siRNA.

RNAi is an evolutionarily conserved, sequence-specific mechanism triggered by double-stranded RNA (dsRNA) that induces degradation of complementary target single stranded mRNA and “silencing” of the corresponding translated sequences (McManus et al., “Gene Silencing in Mammals by Small Interfering RNAs.,” Nature Rev. Genet. 3:737 (2002), which is hereby incorporated by reference in its entirety). RNAi functions by enzymatic cleavage of longer dsRNA strands into biologically active “short-interfering RNA” (siRNA) sequences. SiRNA of the present invention is a short double-stranded RNA composed of 19-22 nucleic acids, which targets mRNA (messenger RNA) of a gene whose nucleotide sequence is identical with its sense strand in order to suppress expression of the gene by decomposing the target gene (Elbashir et al., “Duplexes of 21Nucleotide RNAs Mediate RNA Interference in Cultured Mammalian cells,” Nature 411:494-498 (2001), which is hereby incorporated by reference in its entirety). SiRNA is capable of inhibiting gene expression even with 10 times less amount than the required amount of conventional antisense oligonucleotide and can be used to downregulate or silence the transcription and translation of a gene product of interest, i.e., a target sequence.

Gene silencing of the present invention can be induced by direct transfection of cells with chemically synthesized (Elbashir et al., “Duplexes of 21-Nucleotide RNAs Mediate RNA Interference in Cultured Mammalian Cells,” Nature 411:494-498 (2001), which is hereby incorporated by reference in its entirety) or in vitro transcribed siRNA (Kim et al., “Synthetic dsRNA Dicer Substrates Enhance RNAi Potency and Efficacy,” Nat. Biotechnol. 23:222-226 (2005); Luo et al., “Small Interfering RNA Production by Enzymatic Engineering of DNA (SPEED),” Proc. Natl. Acad. Sci. USA 101:5494-5499 (2004); Myers et al., “Recombinant Dicer Efficiently Converts Large dsRNAs into siRNAs Suitable for Gene Silencing,” Nat. Biotechnol. 21:324-328 (2003), all of which are hereby incorporated by reference in their entirety). Alternatively, it can be obtained by transfecting a plasmid or transducing a viral vector encoding a short hairpin RNA (shRNA) driven by a RNA polymerase (pol) III promoter, including U6, H1, 7SK and tRNA promoters (Brummelkamp et al., “A System for Stable Expression of Short Interfering RNAs in Mammalian Cells,” Science 296:550-553 (2002); Gou et al., “Gene Silencing in Mammalian Cells by PCR-Based Short Hairpin RNA,” FEBS Lett 548:113-118 (2003); Paul et al., “Effective Expression of Small Interfering RNA in Human Cells,” Nat. Biotechnol. 20:505-508 (2002); Sui et al., “A DNA Vector-Based RNAi Technology to Suppress Gene Expression in Mammalian Cells,” Proc. Natl. Acad. Sci. USA 99:5515-5520 (2002), all of which are hereby incorporated by reference in their entirety) or a pol II promoter such as CMV or SP-C (Gou et al., “Gene Silencing in Alveolar type II Cells using Cell-Specific Promoter in Vitro and in Vivo,” Nucleic Acids Res. 32:e134 (2004) and Stegmeier et al., “A Lentiviral MicroRNA-Based System for Single-Copy Polymerase II-Regulated RNA Interference in Mammalian Cells,” Proc. Natl. Acad. Sci. USA 102:13212-13217 (2005), which are hereby incorporated by reference in their entirety). ShRNAs consist of short inverted repeats separated by a small loop sequence and is rapidly processed by the cellular machinery into 19-22 siRNA, thereby suppressing the target gene expression (see, e.g., U.S. Patent Publication No. 2009/0087910 to Liu et al., which is hereby incorporated by reference in its entirety).

MiRNA (microRNAs) in accordance with the present invention are small (approximately 21-22 nucleotides in length, these are also known as “mature” miRNA), non-coding RNA molecules encoded in the genomes of plants and animals. These highly conserved, endogenously expressed RNAs regulate the expression of genes by binding to the 3′-untranslated regions (3′-UTR) of specific mRNAs. MiRNAs may act as key regulators of cellular processes such as cell proliferation, cell death (apoptosis), metabolism, and cell differentiation. On a larger scale, miRNA expression has been implicated in early development, brain development, disease progression (such as cancers and viral infections). There is speculation that in higher eukaryotes, the role of miRNAs in regulating gene expression could be as important as that of transcription factors. More than 200 different miRNAs have been identified in plants and animals (Ambros et al., “MicroRNAs and Other Tiny Endogenous RNAs in C. elegans,” Curr. Biol. 13:807-818 (2003), which is hereby incorporated by reference in its entirety). Mature miRNAs appear to originate from long endogenous primary miRNA transcripts (also known as pri-miRNAs, pri-mirs, pri-miRs or pri-pre-miRNAs) that are often hundreds of nucleotides in length (Lee, et al., “MicroRNA Maturation: Stepwise Processing and Subcellular Localization,” EMBO J. 21(17):4663-4670 (2002), which is hereby incorporated by reference in its entirety).

An antisense nucleic acid can be designed such that it is complementary to the entire coding region of Dlgh1 mRNA, but more preferably is an oligonucleotide that is antisense to only a portion of the coding or noncoding region of Dlgh1 mRNA. For example, the antisense oligonucleotide can be complementary to the region surrounding the translation start site of Dlgh1 mRNA, e.g., between the −10 and +10 regions of the target gene nucleotide sequence. An antisense oligonucleotide can be, for example, about 15, 20, 25, 30, 35, 40, 45, 50, 55, 60, 65, 70, 75, 80, or more nucleotides in length.

An antisense or RNAi nucleic acid of the invention can be constructed using chemical synthesis and enzymatic ligation reactions with procedures known in the art. For example, an antisense or RNAi nucleic acid can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and modified nucleotides can be used. Examples of modified nucleotides which can be used to generate the modified RNAi nucleic acids include 5-fluorouracil, 5-bromouracil, 5-chlorouracil, 5-iodouracil, hypoxanthine, xanthine, 4-acetylcytosine, 5-carboxyhydroxylmethyluracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N⁶-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 5-methyl-2-thiouracil, 3-(3-amino-3-N-2-carboxypropyl) uracil, and 2,6-diaminopurine.

An important feature of RNAi affected by siRNA is the double stranded nature of the RNA and the absence of large overhanging pieces of single stranded RNA, although dsRNA with small overhangs and with intervening loops of RNA has been shown to effect suppression of a target gene. As used herein, it will be understood that the terms siRNA and RNAi are interchangeable. Furthermore, as is well-known in the field, RNAi technology may be effected by siRNA, miRNA, shRNA, or other RNAi inducing agents. Although siRNA will be referred to in general in the specification, it will be understood that any other RNA interfering agents may be used, including shRNA, miRNA, or an RNAi-inducing vector whose presence within a cell results in production of an siRNA, shRNA, or miRNA targeted to a Dlgh1 transcript.

RNA interference is a multistep process and is generally activated by double-stranded RNA (dsRNA) that is homologous in sequence to the targeted Dlgh1 gene. Introduction of long dsRNA into the cells of organisms leads to the sequence-specific degradation of homologous gene transcripts. The long dsRNA molecules are metabolized to small (e.g., 21-23 nucleotide (nt)) interfering RNAs (siRNAs) by the action of an endogenous ribonuclease known as Dicer. The siRNA molecules bind to a protein complex, termed RNA-induced silencing complex (RISC), which contains a helicase activity and an endonuclease activity. The helicase activity unwinds the two strands of RNA molecules, allowing the antisense strand to bind to the targeted Dlgh1 mRNA molecule. The endonuclease activity hydrolyzes the Dlgh1 mRNA at the site where the antisense strand is bound. Therefore, RNAi is an antisense mechanism of action, as a single stranded (ssRNA) RNA molecule binds to the target Dlgh1 mRNA molecule and recruits a ribonuclease that degrades the Dlgh1 mRNA.

An RNAi-inducing agent or RNAi molecule is used in the present application includes for example, siRNA, miRNA, or shRNA targeted to a Dlgh1 transcript or an RNAi-inducing vector whose presence within a cell results in production of a siRNA or shRNA targeted to the target Dlgh1 transcript. Such siRNA or shRNA comprises a portion of RNA that is complementary to a region of the target Dlgh1 transcript. Essentially, the RNAi-inducing agent or RNAi molecule down-regulates expression of the targeted Dlgh1 molecule via RNA interference.

Various delivery methods suitable for the delivery of the RNAi inducing agent (including siRNA, shRNA, miRNA, etc.) may be used. For example, some delivery agents for the RNAi-inducing agents are selected from the following group of cationic polymers, modified cationic polymers, peptide molecular transporters, lipids, liposomes and/or non-cationic polymers. Examples of such polymers include, without limitation, polyethylenimine (e.g., linear or branched PEI) and/or polyethylenimine derivatives, grafted PEIs such as galactose PEI, cholesterol PEI, antibody derivatized PEI, and polyethylene glycol PEI (PEG-PEI) derivatives thereof (see, e.g., Ogris et al., “DNA/Polyethylenimine Transfection Particles: Influence of Ligands, Polymer Size, and PEGylation on Internalization and Gene Expression,” AAPS Pharm. Sci. 3(3):1-11 (2001); Furgeson et al., “Modified Linear Polyethylenimine-Cholesterol Conjugates for DNA Complexation,” Bioconjugate Chem. 14(4):840-847 (2003); Kunath et al., “The Structure of PEG-Modified Poly(ethylene imines) Influences Biodistribution and Pharmacokinetics of their Complexes with NF-kappaB Decoy in Mice,” Pharmaceutical Res. 19(6): 810-817 (2002); Choi et al., “Effect of Poly(ethylene glycol) Grafting on Polyethylenimine as a Gene Transfer Vector In Vitro,” Bull. Korean Chem. Soc. 22:46-52 (2001); Bettinger et al., “Size Reduction of Galactosylated PEI/DNA Complexes Improves Lectin-Mediated Gene Transfer into Hepatocytes,” Bioconjugate Chem. 10:558-561 (1999); Peterson et al., “Polyethylenimine-Graft-Poly(ethylene glycol) Copolymers: Influence of Copolymer Block Structure on DNA Complexation and Biological Activities as Gene Delivery System,” Bioconjugate Chem. 13(4):845-854 (2002); Erbacher et al., J. Gene Medicine Preprint 1:1-18 (1999); Godbey et al., “Tracking the Intracellular Path of Poly(ethylenimine)/DNA Complexes for Gene Delivery,” Proc. Natl. Acad. Sci. USA 96:5177-5181 (1999); Godbey et al., “Poly(ethylenimine) and its Role in Gene Delivery,” J. Control Release 60(2-3):149-160 (1999); Diebold et al., “Mannose Polyethylenimine Conjugates for Targeted DNA Delivery into Dendritic Cells,” J. Biol. Chem. 274:19087-19094 (1999); Thomas and Klibanov, “Enhancing Polyethylenimine's Delivery of Plasmid DNA into Mammalian Cells,” Proc. Natl. Acad. Sci. USA 99(23):14640-14645 (2002); and U.S. Pat. No. 6,586,524 to Sagara, all of which are hereby incorporated by reference in their entirety).

The siRNA molecule of the present invention can also be present in the form of a bioconjugate, for example a nucleic acid conjugate as described in U.S. Pat. No. 6,528,631 to Cook et al., U.S. Pat. No. 6,335,434 to Guzaev et al., U.S. Pat. No. 6,235,886 to Manoharan et al., U.S. Pat. No. 6,153,737 to Manoharan et al., U.S. Pat. No. 5,214,136 to Lin et al., or U.S. Pat. No. 5,138,045 to Cook et al., each of which is hereby incorporated by reference in its entirety.

As a further example, yet another delivery route includes the direct delivery of RNAi inducing agents (including siRNA, shRNA, and miRNA) and even anti-sense RNA (asRNA) via gene constructs followed by the transformation of cells. This results in the transcription of the gene constructs encoding the RNAi inducing agent, such as siRNA, shRNA and miRNA, or even asRNA and provides for the transient or stable expression of the RNAi inducing agent in those transformed cells. Viral vector delivery systems may also be used.

Targeted delivery strategies may also be utilized to deliver the RNAi directly to the cells of interest.

There are a variety of methods for targeting siRNA. RNAi, as a therapeutic modality, has an enormous potential to bring the era of personalized medicine one step further from notion into reality. However, delivery of RNAi effector molecules into their target tissues and cells remain extremely challenging. Major attempts have been made in recent years to develop sophisticated nanocarriers that could overcome these hurdles. Most recent progress has focused mostly on the in vivo RNAi delivery into immune cells. Weinstein et al., “RNAi Nanomedicines: Challenges and Opportunities within the Immune System,” Nanotechnology 21(23):232001 (2010), which is hereby incorporated by reference in its entirety.

RNAi-based approaches have contributed significantly to the improved understanding of gene expression and function in vitro. The ability to apply these strategies in vivo to validate the role of specific genes in normal or pathological conditions, and to induce gene silencing, has led to new possibilities for using RNAi as a novel therapeutic modality. However, the translation of RNAi from an effective genomic tool into clinical applications has been hindered by the challenge of delivering RNAi molecules to their target tissues by systemic administration, particularly to hematopoietic cells. Current systemic RNAi delivery platforms are targeted to leukocytes, with a focus on the integrin-targeted stabilized nanoparticles strategy, which uses leukocyte integrins for the delivery of siRNAs exclusively to cells of the immune system (Elfakess et al., “Overcoming RNAi Transduction in Leukocytes using Targeted and Stabilized Nanoparticles,” IDrugs: The Investigational Drugs Journal 13(9):626-631 (2010), which is hereby incorporated by reference in its entirety).

SiRNA has also proved to be an extremely useful research tool to interrogate gene functions in test tubes. The transformation of siRNAs from a functional genomic tool into a new therapeutic modality has been hindered by ineffective delivery methods for systemic administration. Platforms utilize leukocyte integrins as receptor targets for siRNAs delivery and are utilized for in vivo drug target validation of a novel anti-inflammatory target, cyclin D1, for inflammatory bowel diseases (Peer et al., “Systemic siRNA Delivery to Leukocyte-Implicated Diseases,” Cell Cycle 8(6):853-859 (2009), which is hereby incorporated by reference in its entirety). For example, cyclin D1 (CyD1) is a pivotal cell cycle-regulatory molecule and a well-studied therapeutic target for cancer. Although CyD1 is strongly up-regulated at sites of inflammation, its exact roles in this context were uncharacterized until a recent discovery that CyD1 may be an anti-inflammatory T cell target. In a study by Peer et al. (Peer et al., “Systemic Leukocyte-Directed siRNA Delivery Revealing Cyclin D1 as an Anti-Inflammatory Target,” Science 319(5863):627-630 (2008), which is hereby incorporated by reference in its entirety) CyD1 was selectively silenced in leukocytes in vivo. Targeted stabilized nanoparticles (tsNPs) were loaded with CyD1-siRNA. CyD1 was shown to be a potential anti-inflammatory target, suggesting that the application of similar modes of targeting by siRNA may be feasible in other therapeutic settings as well.

Dlgh1 siRNA can be targeted to Treg to inactivate cells, which may be advantageous in cancer therapy, where Tregs protect the cancer cells from attack by the host immune system. In one study, a protamine-antibody fusion protein was designed to deliver siRNA to HIV-infected or envelope-transfected cells. SiRNAs bound to F105-P induced silencing only in cells expressing HIV-1 envelope and targeted against the HIV-1 capsid gene gag inhibited HIV replication in hard-to-transfect, HIV-infected primary T cells. Administration of F105-P-complexed siRNAs into mice targeted HIV envelope-expressing B16 melanoma cells, but not normal tissue or envelope-negative B16 cells, demonstrating the potential for systemic, cell-type specific, antibody-mediated siRNA delivery (Song et al., “Antibody Mediated In Vivo Delivery of Small Interfering RNAs via Cell-Surface Receptors,” Nature Biotechnology 23(6):709-717 (2005), which is hereby incorporated by reference in its entirety).

In addition to targeting Dlgh1 siRNA to inactivate Treg cells, it is also possible to inhibit PDZ domain bindings. In particular, Dlgh1 has three PDZ domains that could be targeted by such inhibitors. See Mayasundari et al., “Rational Design of the First Small-Molecule Antagonists of NHERF1/EBP50 PDZ Domains,” Bioorganic & Medicinal Chemistry Letters 18(3):942-945 (2008), which is hereby incorporated by reference in its entirety (describing the first small-molecule antagonists that specifically target the ligand-binding pocket of PDZ domains of NHERF1 multi-functional adaptor protein); see also Mahindroo et al., “Indole-2-Amide Based Biochemical Antagonist of Dishevelled PDZ Domain Interaction Down-Regulates Dishevelled-Driven Tcf Transcriptional Activity,” Bioorganic & Medicinal Chemistry Letters 18(3):946-949 (2008), which is hereby incorporated by reference in its entirety (designing a series of indole-2-amide-based compounds that antagonize interaction between the Dishevelled (Dvl) PDZ domain and a peptide derived from the natural PDZ ligand Frizzled-7, which inhibited Tcf-mediated transcription activated by exogenous Dvl via the biochemical antagonism).

The development of inhibitors of Dishevelled (Dvl) PDZ protein-protein interactions has important potential for drug development applications. A tripeptide VVV binds to the PDZ domain of Dvl. The tripeptide may be used as a scaffold to optimize an antagonist for targeting Dvll PDZ protein-protein interaction (Lee et al., “Identification of Tripeptides Recognized by the PDZ Domain of Dishevelled,” Bioorganic & Medicinal Chemistry 17(4):1701-1708 (2009), which is hereby incorporated by reference in its entirety). These methods could produce results that are effective as anti-cancer and anti-infection treatments.

Another aspect of the present invention relates to a method of treating an inflammatory condition in a subject. The method includes selecting a subject with an inflammatory condition and administering to the selected subject an agent which enhances Dlgh1 expression, recruitment to the immunological synapse and/or activity under conditions effective to treat the inflammatory condition. In one embodiment, the subject is selected based on it having an inflammatory condition mediated by Dlgh1 protein expression, recruitment to the immunological synapse, and/or activity under conditions effective to treat the inflammatory condition.

A suitable subject for treatment in accordance with this aspect of the present invention is a subject having or at risk of developing an inflammatory condition. The term “subject” in the context of the present invention refers to any living vertebrate, preferably a human.

As used herein, the inflammatory condition may, in various embodiments, be selected from the group consisting of an autoimmune disease, rheumatoid arthritis, pericarditis, vasculitis, lupus, bronchitis, phrenitis, acute and chronic enterocolitis, ulcerative colitis, inflammatory bowel disease, type I diabetes, multiple sclerosis, psoriasis, inflammatory bowel disease, Crohn's disease, ulcerative cholitis, and autoimmune disorders.

The anti-inflammatory agents of the present aspect can be administered as part of a combination therapy in conjunction with another active agent, depending upon the nature of the infection that is being treated. Such additional active agents include anti-infective agents, antibiotic agents, and antimicrobial agents. The anti-inflammatory agents, anti-infective agents, antibiotic agents, and antimicrobial agents may be combined prior to administration, or administered concurrently (as part of the same composition or by way of a different composition) or sequentially with the inventive therapeutic compositions of the present invention. In certain embodiments, the administering is repeated. The subject may be an infant, juvenile, or adult.

Anti-inflammatory agents that may be used in the present invention may be, but are not limited to, non-steroidal anti-inflammatory drugs (NSAID). Examples of NSAIDs that may be used in accordance with the present invention include any locally or systemically active non-steroidal anti-inflammatory drugs which can be delivered through the skin. Suitable non-steroidal anti-inflammatory drugs include ibuprofen, flurbiprofen, ketoprofen, aclofenac, diclofenac, aloxiprin, aproxen, aspirin, diflunisal, fenoprofen, indomethacin, mefenamic acid, naproxen, phenylbutazone, piroxicam, salicylamide, salicylic acid, sulindac, desoxysulindac, tenoxicam, tramadol, ketoralac, acemetacin, amtolmetin, azapropazone, benorilate, benoxaprofen, benzydamine hydrochloride, bromfenal, bufexamac, butibufen, carprofen, celecoxib, choline salicylate, clonixin, dipyone, droxicam, etodolac, etofenamate, etoricoxib, felbinac, fenbufen, fentiazac, floctafenine, flufenamic acid, indoprofen, isoxicam, lornoxicam, loxoprofen, licofelone, fepradinol, magnesium salicylate, meclofenamic acid, meloxicam, morniflumate, niflumic acid, nimesulide, oxaprozen, piketoprofen, priazolac, pirprofen, propyphenazone, proquazone, rofecoxib, salalate, sodium salicylate, sodium thiosalicylate, suprofen, tenidap, tiaprofenic acid, tolmetin, trolamine salicylate, and zomepirac. The non-steroidal anti-inflammatory agent may be a racemic mixture or individual enantiomers where applicable.

Alternatively, the anti-inflammatory agents of the present invention may be steroidal anti-inflammatory agents. Examples of steroidal anti-inflammatory agents that may be utilized in the present invention include, but are not limited to, cortisone, betamethasone, dexamethasone, fluprednisolone, hydrocortisone, methylprednisolone, paramethasone, prednisolone, prednisone and triamcinolone. Compositions may contain a combination of steroidal anti-inflammatory agents, non-steroidal anti-inflammatory agents, or both-steroidal and non-steroidal anti-inflammatory agents.

As used herein, the term agent refers to a compound which enhances Dlgh1 expression, recruitment to the immunological synapse and/or activity under conditions effective to treat the inflammatory condition. In one embodiment, the agent is selected from the group consisting of inhibitors of tumor necrosis factor-α (TNF-α) effects on Dlgh1 recruitment to the immunological synapse and direct activators of p38 mitogen activated kinase action on nuclear factor of activated T cells.

Tumor necrosis factor-α (also called TNF-α, TNFα, cachexin, or cachetin), as used herein, stimulates T cells that coordinate the immune system. In a healthy human body, TNF-α is released by white blood cells and other tissues in response to damage caused by an infection. TNF-α is released from injured or herniated disks. During an inflammatory response, nerve cells communicate with each other by releasing neuro-transmitter glutamate. This process follows activation of a nerve cell receptor called CXCR4 by the inflammatory mediator stromal cell-derived factor 1 (SDF-1). An extraordinary feature of the nerve cell communication is the rapid release of inflammatory mediator TNF-α. Subsequent to release of TNF-α, there is an increase in the formation of inflammatory mediator prostaglandin. Excessive prostaglandin release results in an increased production of neurotransmitter glutamate and an increase in nerve cell communication resulting in a vicious cycle of inflammation. There is excitation of pain receptors and stimulation of the specialized nerves e.g., C fibers and A-delta fibers that carry pain impulses to the spinal cord and brain.

Protein kinases related to the MAPK pathway (e.g., p38 MAPK, JNK, ERK) are therapeutic targets for both inflammatory diseases (see, e.g., Kaminska, “MAPK Signalling Pathways as Molecular Targets for Anti-Inflammatory Therapy—From Molecular Mechanisms to Therapeutic Benefits,” Biochim. Biophys. Acta. 1754:253-262 (2005); Kumar et al., “P38 MAP Kinases: Key Signalling Molecules as Therapeutic Targets for Inflammatory Diseases,” Nat. Rev. Drug Discov. 2(9):717-726 (2003); Saklatvala, “The p38 MAP Kinase Pathway as a Therapeutic Target in Inflammatory Disease,” Curr. Opin. Pharmacol. 4(4):372-377 (2004), which are hereby incorporated by reference in their entirety) and cancer (see, e.g., Engelberg, “Stress-Activated Protein Kinases-Tumor Suppressors or Tumor Initiators?,” Sem. Cancer Biol. 14(4):271-282 (2004); Platanias, “Map Kinase Signaling Pathways and Hematologic Malignancies,” Blood 101(12):4667-4679 (2003); Rennefahrt et al., “Stress Kinase Signaling in Cancer: Fact or Fiction?,” Cancer Lett. 217(1):1-9 (2005), each which is hereby incorporated by reference in its entirety). The p38 MAPK family consists of at least four isoforms, p38α, β (and P2), δ, γ, which are encoded by separate genes, expressed in different tissues and may have distinct functions (U.S. Patent Publication No. 2010/0016587 to Watterson et al., which is hereby incorporated by reference in its entirety).

In terms of p38 mitogen activated kinase's (p38 MAPK) role in inflammatory diseases, activation of p38 MAPK has been shown to regulate gene expression and lead to increased production of proinflammatory cytokines (see, e.g., Schieven, “The Biology of p38 Kinase: A Central Role in Inflammation,” Curr. Topics Med. Chem. 5(10):921-928 (2005), which is hereby incorporated by reference in its entirety). The mechanisms by which p38 MAPK stimulates proinflammatory cytokine production include phosphorylation and activation of transcription factors, some of which can increase transcription of inflammatory cytokine genes; regulation of cytokine mRNA stability and translation; and regulation of transcriptional activation of certain cytokines Thus, p38 MAPK can modulate a number of different signaling events that can converge on proinflammatory cytokine up-regulation. (U.S. Patent Publication No. 2010/0016587 to Watterson et al., which is hereby incorporated by reference in its entirety).

According to the present methods, the term activator of p38 mitogen activated kinase means any material that promotes signaling through the p38 MAP kinase pathway. p38 MAPK has been found to be a component of tumor suppressor pathways under some conditions and as a pro-oncogenic component under other conditions (see, e.g., Engelberg, “Stress-Activated Protein Kinases-Tumor Suppressors or Tumor Initiators?,” Sem. Cancer Biol. 14(4):271-282 (2004); Rennefahrt et al., “Stress Kinase Signaling in Cancer: Fact or Fiction?,” Cancer Lett. 217(1):1-9 (2005), which are hereby incorporated by reference in their entirety). The kinase has been shown to be involved in cell growth, differentiation, cell cycle control, and apoptosis. In certain cancers, p38 MAPK is activated and mediates cell proliferation. In addition, p38 MAPK is activated in response to environmental stresses and damaging agents, such as UV irradiation, which can induce tumors (see, e.g., Jinlian, “P38 MAPK in Regulating Cellular Responses to Ultraviolet Radiation,” J. Biomed. Sci. 14(3):303-312 (2007); U.S. Patent Publication No. 2010/0016587 to Watterson et al., which are hereby incorporated by reference in their entirety).

Examples of p38 MAPK kinase include RK, Mxi-2, CSBP1/2 or HOG-1-related kinases. P38 MAPK is primarily activated by cellular stresses, including heat and osmotic shock, UV irradiation, proinflammatory cytokines, and hypoxia/reoxygenation (Howe et al., “Activation of the MAP Kinase Pathway by the Protein Kinase Raf,” Cell 71:335-342 (1992); Kyriakis et al., “The Stress-Activated Protein Kinase Subfamily of c-Jun Kinases,” Nature 369:156-160 (1994); Derijard et al., “JNK1: A Protein Kinase Stimulated by UV Light and Ha-Ras that Binds and Phosphorylates the c-Jun Activation Domain,” Cell 76:1025-1037 (1994); Minden et al., “c-Jun N-Terminal Phosphorylation Correlates with Activation of the JNK Subgroup but not the ERK Subgroup of Mitogen-Activated Protein Kinases,” Mol. Cell. Biol. 14:6683-6688 (1994); Raingeaud et al., “Pro-Inflammatory Cytokines and Environmental Stress Cause p38 Mitogen-Activated Protein Kinase Activation by Dual Phosphorylation on Tyrosine and Threonine,” J. Biol. Chem. 270:7420-7426 (1995); Han et al., “A MAP Kinase Targeted by Endotoxin and Hyperosmolarity in Mammalian Cells,” Science 265: 808-811 (1994); Gupta et al., “Selective Interaction of JNK Protein Kinase Isoforms with Transcription Factors,” EMBO J. 15:2760-2770 (1996), all of which are hereby incorporated by reference in their entirety). Four isoforms of p38 MAPK have been identified in mammalian cells (Lee et al., “A Protein Kinase Involved in the Regulation of Inflammatory Cytokine Biosynthesis,” Nature 372:739-746 (1994); Rouse et al., “A Novel Kinase Cascade Triggered by Stress and Heat Shock that Stimulates MAPKAP Kinase-2 and Phosphorylation of the Small Heat Shock Proteins,” Cell 78:1027-1037 (1994); Jiang et al., “Characterization of the Structure and Function of a New Mitogen-Activated Protein Kinase (p38beta),” J. Biol. Chem. 271:17920-17926 (1996); Lechner et al., “ERK6, a Mitogen-Activated Protein Kinase Involved in C2C12 Myoblast Differentiation,” Proc. Natl. Acad. Sci. USA 93:4355-4359 (1996); Li et al., “The Primary Structure of P38 Gamma: A New Member of P38 Group of MAP Kinases,” Biochem. Biophys. Res. Commun. 228: 334-340 (1996); Mertens et al., “SAP Kinase-3, A New Member of the Family of Mammalian Stress-Activated Protein Kinases,” FEBS Lett. 383:273-276 (1996); Cuenda et al., “Activation of Stress-Activated Protein Kinase-3 (SAPK3) by Cytokines and Cellular Stresses is Mediated via SAPKK3 (MKK6); Comparison of the Specificities of SAPK3 and SAPK2 (RK/p38),” EMBO J. 16: 295-305 (1997); Wang et al., “Molecular Cloning and Characterization of a Novel p38 Mitogen-Activated Protein Kinase,” J. Biol. Chem. 272:23668-23674 (1997); Enslen et al. “Selective Activation of P38 Mitogen-Activated Protein (MAP) Kinase Isoforms by the MAP Kinase Kinases MKK3 and MKK6,” J. Biol. Chem. 273:1741-1748 (1998), all of which are hereby incorporated by reference in their entirety).

No physiological role has been associated with the difference in substrate affinity of the p38 MAPK. Kinase 4 (MKK4/SEK-1), MKK7, MKK3, and MKK6 have been identified as activators of p38 MAPK, displaying some degree of selectivity for individual p38 MAPK isoforms (Enslen et al. “Selective Activation of P38 Mitogen-Activated Protein (MAP) Kinase Isoforms by the MAP Kinase Kinases MKK3 and MKK6,” J. Biol. Chem. 273:1741-1748 (1998); Derijard et al., “Independent Human MAP-Kinase Signal Transduction Pathways Defined by MEK and MKK Isoforms,” Science 267:682-685 (1995); Lin et al., “Identification of a Dual Specificity Kinase that Activates the Jun Kinases and P38-Mpk2,” Science 268:286-290 (1995); Sanchez et al., “Role of SAPK/ERK Kinase-1 in the Stress-Activated Pathway Regulating Transcription Factor c-Jun,” Nature 372:794-798 (1994); Moriguchi et al., “A Novel Kinase Cascade Mediated by Mitogen-Activated Protein Kinase Kinase 6 and MKK3,” J. Biol. Chem. 271:13675-13679 (1996); Holland et al., “MKK7 is a Stress-Activated Mitogen-Activated Protein Kinase Kinase Functionally Related to Hemipterous,” J. Biol. Chem. 272:24994-24998 (1997); Raingeaud et al., “MKK3- and MKK6-Regulated Gene Expression is Mediated by the P38 Mitogen-Activated Protein Kinase Signal Transduction Pathway,” Mol. Cell. Biol. 16:1247-1255 (1996), all of which are hereby incorporated by reference in their entirety). MKK6 functions as an activating kinase for all known p38 MAPK isoforms, whereas MKK3 predominantly activates the isoform p38 MAPK δ. Among the identified substrates of mitogen-activated protein kinases are a variety of transcription factors that become activated upon their phosphorylation (Marshall, C. J., “Specificity of Receptor Tyrosine Kinase Signaling: Transient Versus Sustained Extracellular Signal-Regulated Kinase Activation,” Cell 80:179-185 (1995); Robinson et al., “Mitogen-Activated Protein Kinase Pathways,” Curr. Opin. Cell Biol. 9:180 (1997); Cano et al., “Parallel Signal Processing Among Mammalian MAPKs,” Trends Biochem. Sci. 20:117-122 (1995); Treisman, R. “Regulation of Transcription by MAP Kinase Cascades,” Curr. Opin. Cell. Biol. 8:205-215 (1996), all of which are hereby incorporated by reference in their entirety).

Because of its importance in modulating proinflammatory cytokine production, p38 MAPK is a compelling therapeutic target for small molecule development against inflammatory diseases characterized by elevated levels of proinflammatory cytokines In addition, because of the increasing awareness of the pro-oncogenic potential of p38 MAPK and its observed effects in mediating a number of cellular processes important for cancer onset and progression, targeting p38 MAPK is a therapeutic option for treating cancer, especially for hematologic malignancies (see, e.g., Platanias, “Map Kinase Signaling Pathways and Hematologic Malignancies,” Blood 101(12):4667-4679 (2003), which is hereby incorporated by reference in its entirety). The present invention is not limited to a particular mechanism. Indeed, an understanding of the mechanism is not necessary to practice the present invention. Nonetheless, it is contemplated that the inhibition of p38 MAPK is a useful therapy for diseases and/or disorders where p38 MAPK is over-expressed or over-activated. (U.S. Patent Publication No. 2010/0016587 to Watterson et al., which is hereby incorporated by reference in its entirety).

In one embodiment, progression of the condition in the selected subject is monitored. Effectiveness of treatment may be monitored by comparing detected levels of inflammation in a sample at various times, including during initial testing and prior diagnosis, as well as prior to and after an initial treatment, and/or prior to and after each subsequent treatment. Effectiveness may also be determined based on measurements after treatment ends. This method may be performed on a sample which is a biological fluid. Examples of such biological fluids include blood, plasma, serum, urine, spinal fluid, synovial fluid, amnionic fluid, and cranial fluid. In one embodiment, wherein the monitoring indicates progression of the condition in the selected subject, the method further comprises administering to the selected subject the agent which enhances Dlgh1 expression, recruitment to the immunological synapse, and/or activity to delay progression of the condition.

In another embodiment, the monitoring includes measuring expression, recruitment to the immunological synapse, and/or activity of Dlgh1 in CD4⁺CD25⁻ regulatory T cells in the subject before and after said administering and comparing the Dlgh1 expression, recruitment to the immunological synapse, and/or activity before said administering to the Dlgh1 expression, recruitment to the immunological synapse, and/or activity after said administering, where progression of the subject's inflammatory condition is determined based on said comparing. In another embodiment, the measuring further includes contacting a sample of Dlgh1 in CD4⁺CD25⁺ regulatory T cells from the selected subject with a reagent which recognizes Dlgh1 expression, recruitment to the immunological synapse, and/or activity.

According to the present invention, the monitoring can begin before initial treatment, as well as at a time before any subsequent treatments. Therefore, the sample may be obtained prior to initial treatment and prior to any or all subsequent treatments. In one embodiment, the method can include developing a follow-up treatment regimen based on said comparing.

An additional aspect of the present invention relates to a method of treating cancer in a subject. The method includes selecting a subject with a cancer and administering to the selected subject an agent which inhibits Dlgh1 protein expression, recruitment to the immunological synapse, and/or activity in CD4⁺CD25⁺ regulatory T cells under conditions effective to treat the cancer. In one embodiment, the subject is selected based on it having a cancer mediated by Dlgh1 protein expression, recruitment to the immunological synapse, and/or activity in CD4⁺CD25⁺ regulatory T cells under conditions effective to treat the cancer.

Examples of types of cancer that can be treated include acute lymphocytic leukemia, acute myelogenous leukemia, biliary cancer, breast cancer, cervical cancer, chronic lymphocytic leukemia, chronic myelogenous leukemia, colorectal cancer, endometrial cancer, esophageal cancer, gastric cancer, head and neck cancer, Hodgkin's lymphoma, lung cancer, medullary thyroid cancer, non-Hodgkin's lymphoma, multiple myeloma, renal cancer, prostate cancer, glial and other brain and spinal cord tumors, pancreatic cancer, melanoma, ovarian cancer, liver cancer, and urinary bladder cancer. The chemotherapeutic compounds of the present method may inhibit Dlgh1 expression and recruitment to the immunological synapse or activity in the Treg cells and are in accordance with those chemotherapeutic compounds described supra. The chemotherapeutic agents of the present aspect can be administered as part of a combination therapy in conjunction with another active agent, as described in detailed supra. The chemotherapeutic agents may be combined prior to administration, or administered concurrently (as part of the same composition or by way of a different composition) or sequentially with the inventive therapeutic compositions of the present invention. In certain embodiments, the administering is repeated. The subject may be an infant, juvenile, or adult.

In one embodiment, the agent is selected from the group consisting of tumor necrosis factor-α (TNF-α) and p38 inhibitors acting on CD4⁺CD25⁺ regulatory T cells as described supra. TNF-α has been reported as being involved in the proliferation of cells, especially deformed cells and cancerous cells.

In one embodiment, the agent reduces or silences Dlgh1 protein expression consistent with those methods described supra. In another embodiment, the progression of the cancer in the selected subject is monitored. This monitoring is carried out in accordance with the previously described aspects of the invention. In yet another embodiment, when the monitoring indicates progression of the cancer in the selected subject, the method further includes administering to the selected subject the agent which inhibits Dlgh1 protein expression, recruitment to the immunological synapse, and/or activity in CD4⁺ CD25⁺ regulatory T cells to delay progression of the cancer.

A further aspect of the present invention relates to a method of treating an infectious disease in a subject. The method includes selecting a subject with an infectious disease and administering to the selected subject an agent which inhibits Dlgh1 protein expression and/or activity under conditions effective to treat the infectious disease. In one embodiment, the subject is selected based on it having an infectious disease mediated by Dlgh1 protein expression and/or activity under conditions effective to treat the infectious disease.

In the present context, the term anti-infective agent covers agents that are capable of killing, inhibiting or otherwise slowing the growth of the infectious agent. The term anti-infective agent may be used interchangeably with the term antibiotic or anti-viral agent or anti-fungal agent, depending on the nature of the infectious agent.

Examples of infectious diseases suitable for treatment by the present method include, but are not limited to bacterial, viral, parasitic, fungal, helminthic, and prion infections. This aspect of the present invention is carried out with regard to methods as described supra.

The therapeutic agents of the present invention can be administered as part of a combination therapy in conjunction with another active agent, depending upon the nature of the infection that is being treated. Such active agents include anti-infective agents, antibiotic agents, and antimicrobial agents. As used herein, an effective amount of an anti-infective agent is an amount that prevents, reduces the severity of, or delays an infection. Examples of anti-infective agents that may be useful in the present invention include, but are not limited to, vancomycin and lysostaphin. In certain embodiments, the administering is repeated. The progression of the disease in the selected subject may be monitored in accordance with the previously described aspects.

Effective doses of the agents and compositions of the present invention, for the treatment of the above described conditions vary depending upon many different factors, including means of administration, target site, physiological state of the patient, other medications administered, and whether treatment is prophylactic or therapeutic. Treatment dosages need to be titrated to optimize safety and efficacy.

Therapeutic agents and compositions of the present invention may be administered in a single dose, or in accordance with a multi-dosing protocol. For example, relatively few doses of the therapeutic composition are administered, such as one or two doses. However, the different dosages, timing of dosages, and relative amounts of the therapeutic agent can be selected and adjusted by one of ordinary skill in the art. The compounds of the present invention may be administered alone or in combination with suitable pharmaceutical carriers or diluents. The diluent or carrier ingredients should be selected so that they do not diminish the therapeutic effects of the compounds of the present invention.

Agents of the present invention are often administered as pharmaceutical compositions comprising an active therapeutic agent and a variety of other pharmaceutically acceptable components. See Remington's Pharmaceutical Science (15th ed., Mack Publishing Company, Easton, Pa., 1980), which is hereby incorporated by reference in its entirety. The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

Pharmaceutical compositions can also include large, slowly metabolized macromolecules, such as proteins, polysaccharides like chitosan, polylactic acids, polyglycolic acids and copolymers (e.g., latex functionalized sepharose, agarose, cellulose, and the like), polymeric amino acids, amino acid copolymers, and lipid aggregates (e.g., oil droplets or liposomes). Additionally, these carriers can function as immuno stimulating agents (i.e., adjuvants).

Agents of the present invention can be administered by parenteral, topical, intravenous, oral, subcutaneous, intraperitoneal, intranasal or intramuscular means for prophylactic and/or therapeutic treatment. The most typical route of administration is subcutaneous although others can be equally effective. The next most common is intramuscular injection. This type of injection is most typically performed in the arm or leg muscles. Intravenous injections as well as intraperitoneal injections, intra-arterial, intracranial, or intradermal injections are also effective in generating a response. The compounds may be administered alone or with suitable pharmaceutical carriers, and can be in solid or liquid form such as tablets, capsules, powders, solutions, suspensions, or emulsions.

An emulsion is a formulation that contains water and oil and is stabilized with an emulsifier. These include lipophilic creams, which are called water-in-oil emulsions, and hydrophilic creams, which are called oil-in-water emulsions. The cream base for water-in-oil emulsions are normally absorption bases such as vaseline, ceresin or lanolin. The bases for oil-in-water emulsions are generally mono-, di- and triglycerides of fatty acids or fatty alcohols with soaps, alkyl sulphates or alkyl polyglycol ethers as emulsifiers.

Carriers include polymeric vehicles including, without limitation, poly(ethylene-co-vinyl acetate), poly-L-lactide, poly-D-lactide, polyglycolide, poly(lactide-co-glycolide), polyanhydride, polyorthoester, polycaprolactone, polyphospagene, proteinaceous polymer, polyether, silicone or combinations thereof.

Another suitable type of carrier is a hydrogel matrix. Hydrogels are of special interest in biological environments since they have high water content as is found in body tissue and are highly biocompatible. Hydrogels and natural biological gels have hydrodynamic properties similar to that of cells and tissues. Hydrogels minimize mechanical and frictional irritation to the surrounding tissue because of their soft and compliant nature. Therefore, hydrogels provide a user-friendly delivery vehicle.

Two classes of biodegradable hydrogels have been developed for controlled release of a wide range of bioactive agents. See Kim et al., “Synthesis and Characterization of Dextran-Methacrylate and its Structure Study by SEM,” J. Biomed. Mater. Res. 49(4):517 (2000) and Park et al., “Biodegradable Hydrogels for Drug Delivery,” Technomic (1993), which are hereby incorporated by reference in their entirety). These biodegradable hydrogels are synthesized from dextran, a naturally occurring biodegradable, biocompatible, and hydrophilic polysaccharide, and synthetic biodegradable hydrophobic polymers, such as polylactides. Both dextran and synthetic biodegradable polyesters like polyglycolide, polylactide, and their copolymers are FDA approved raw biomaterials that are commercially successful as synthetic, absorbable polymers for biomedical uses, e.g., as wound closure devices. The degradation products of PGA and PLA are natural metabolites and are readily eliminated by the human body. The use of these materials in a delivery vehicle is described, for example, in U.S. Application Publ. No. 2006/0240071 to Lerner et al., which is hereby incorporated by reference in its entirety.

A further type of carrier is a buccal bioadhesive formulation, a number of which are known in the art. One exemplary buccal bioadhesive formulation, known as GelClair™, includes water, maltodextrin, propylene glycol, polyvinylpyrrolidone, sodium hyaluronate, potassium sorbate, sodium benzoate, hydroxyethylcellulose, PEG-40 hydrogenated castor oil, disodium edetate, benzalkonium chloride, flavoring, sodium saccharin, and glycyrrhetinic acid.

Yet another form of carrier is a liposomal delivery vehicle, a number of which are known in the art.

Suitable dosage forms for oral use include tablets, dispersible powders, granules, capsules, suspensions, syrups, and elixirs. Inert diluents and carriers for tablets include, for example, calcium carbonate, sodium carbonate, lactose, and talc. Tablets may also contain granulating and disintegrating agents such as starch and alginic acid, binding agents such as starch, gelatin, and acacia, and lubricating agents such as magnesium stearate, stearic acid, and talc. Tablets may be uncoated or may be coated by known techniques to delay disintegration and absorption. Inert diluents and carriers which may be used in capsules include, for example, calcium carbonate, calcium phosphate, and kaolin. Suspensions, syrups, and elixirs may contain conventional excipients, for example, methyl cellulose, tragacanth, sodium alginate; wetting agents, such as lecithin and polyoxyethylene stearate; and preservatives, e.g., ethyl-p-hydroxybenzoate.

Dosage forms suitable for the present invention may be formulated for parenteral administration. For parenteral administration, agents of the present invention can be administered as injectable dosages of a solution or suspension of the substance in a physiologically acceptable diluent with a pharmaceutical carrier that can be a sterile liquid such as water, oil, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin. Peanut oil, soybean oil, and mineral oil are all examples of useful materials. In general, glycols, such as propylene glycol or polyethylene glycol, are preferred liquid carriers, particularly for injectable solutions. Under ordinary conditions of storage and use, these preparations contain a preservative to prevent the growth of microorganisms.

Formulations suitable for injectable use include sterile aqueous solutions or dispersions and sterile powders for the extemporaneous preparation of sterile injectable solutions or dispersions. The preparation also can be emulsified or encapsulated in liposomes or micro particles, such as polylactide, polyglycolide, or copolymer, for enhanced adjuvant effect (Langer et al., “New Methods of Drug Delivery,” Science 249:1527 (1990) and Hanes et al., “New Advances in Microsphere-Based Single-Dose Vaccines,” Advanced Drug Delivery Reviews 28:97-119 (1997), which are hereby incorporated by reference in their entirety). In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

When it is desirable to deliver the agents of the present invention systemically, they may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Examples of parenteral administration are intraventricular, intracerebral, intramuscular, intravenous, intraperitoneal, rectal, and subcutaneous administration. Additional formulations suitable for other modes of administration include oral, intranasal, and pulmonary formulations, suppositories, and transdermal applications.

Intraperitoneal or intrathecal administration of the agents of the present invention can also be achieved using infusion pump devices. Such devices allow continuous infusion of desired compounds avoiding multiple injections and multiple manipulations.

For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%-70%.

For transdermal or intradermal delivery, topical administration can be facilitated by co-administration of the agent with cholera toxin or detoxified derivatives or subunits thereof or other similar bacterial toxins (See Glenn et al., “Skin Immunization Made Possible by Cholera Toxin,” Nature 391:851 (1998), which is hereby incorporated by reference in its entirety). Co-administration can be achieved by using the components as a mixture or as linked molecules obtained by chemical crosslinking or expression as a fusion protein.

Alternatively, transdermal delivery can be achieved using a skin path or using transferosomes (Paul et al., “Transdermal Immunization With Large Proteins by Means of Ultradeformable Drug Carriers,” Eur. J. Immunol. 25:3521-24 (1995); Cevc et al., “Ultraflexible Vesicles, Transfersomes, Have an Extremely Low Pore Penetration Resistance and Transport Therapeutic Amounts of Insulin Across the Intact Mammalian Skim,” Biochem. Biophys. Acta 1368:201-15 (1998), which are hereby incorporated by reference in their entirety). Exemplary modes of transdermal administration include, without limitation, topically to the skin or mucosa, such as those of the nose, throat, gastrointestinal tract, upper airway, oral cavity, anogenital region. Preferred routes of transdermal administration include as an oral rinse, suspension, emulsion, cream or gel suitable for application to the skin.

In addition to the formulations described previously, the agents may also be formulated as a depot preparation. Such long acting formulations may be formulated with suitable polymeric or hydrophobic materials (for example, as an emulsion in an acceptable oil) or ion exchange resins, or as sparingly soluble derivatives (for example, as a sparingly soluble salt).

Another aspect of the present invention relates to a method of inhibiting regulatory T cell activity. The method includes administering to the regulatory T cells an agent that inhibits Dlgh1 protein expression under conditions effective to inhibit regulatory T cell activity. This aspect is carried out with regard to methods described supra.

### EXAMPLES

The following examples are provided to illustrate embodiments of the present invention but are by no means intended to limit its scope.

### Example 1

### Materials and Methods

Cell Purification—

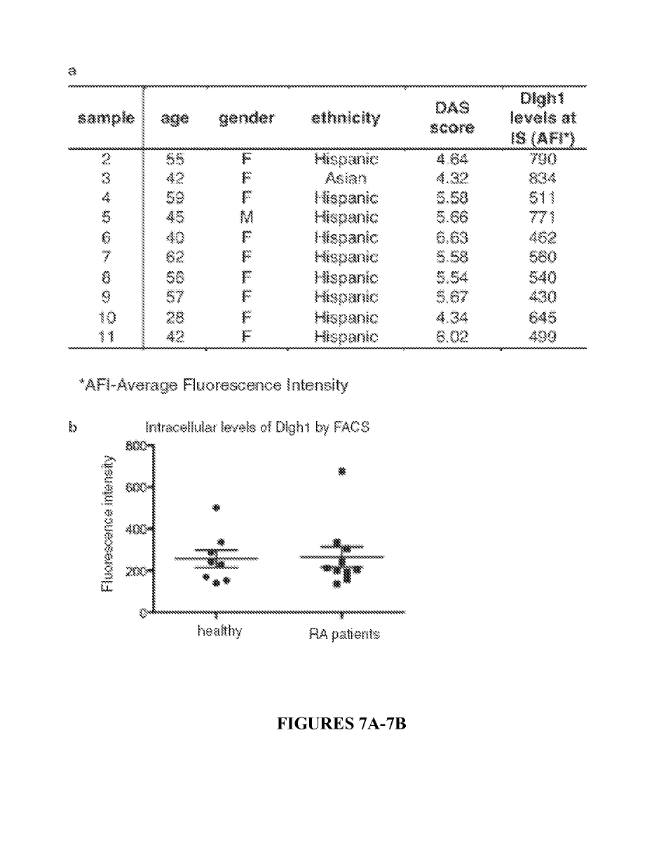

CD4⁺CD25^{hi}, CD4⁺CD25⁺, and CD4⁺CD25⁻ T cells were purified from the peripheral blood of healthy human donors between the ages of 16 and 75 years (Zanin-Zhorov et al., “Protein Kinase C-Theta Mediates Negative Feedback on Regulatory T Cell Function,” Science 328:372-376 (2010), which is hereby incorporated by reference in its entirety) (New York Blood Center) or from 10 patients with RA in different stages (according to DAS; FIG. 7A) as described (Prevoo et al., “Modified Disease Activity Scores That Include Twenty-Eight-Joint Counts. Development and Validation in a Prospective Longitudinal Study of Patients With Rheumatoid Arthritis,” Arthritis Rheum. 38:44-48 (1995), which is hereby incorporated by reference in its entirety). The New York University Institutional Review Board has reviewed the use of human specimens for this study. UCB CD25⁺ and CD25 CD4⁺ T cells were isolated from frozen UCB units (National Placental Blood Program, New York Blood Center) by positive selection using directly conjugated anti-CD25 magnetic microbeads and expanded as described (Hippen et al., “Umbilical Cord Blood Regulatory T-Cell Expansion and Functional Effects of Tumor Necrosis Factor Receptor Family Members OX40 and 4-1BB Expressed on Artificial Antigen-Presenting Cells,” Blood 112:2847-2857 (2008), which is hereby incorporated by reference in its entirety).

Planar Lipid Bilayers—

Planar lipid bilayers containing anti-CD3 antibodies (5 μg/mL) and ICAM-1 (250 molecules per mm²) were prepared in parallel-plate flow cells as described (Zanin-Zhorov et al., “Protein Kinase C-Theta Mediates Negative Feedback on Regulatory T Cell Function,” Science 328:372-376 (2010), which is hereby incorporated by reference in its entirety). The flow cell containing the bilayers was warmed up to 37° C.; cells were injected in 500 μL of Hepes-buffered saline containing 1% human serum albumin; and images were collected on a custom automated Nikon inverted fluorescence microscope.

Microscopy—

All TIRF imaging was performed on the custom automated Nikon inverted fluorescence microscope using the 100×/1.45 N.A. TIRF objective from Nikon. TIRF illumination was set up and aligned according to the manufacturer's instructions as described (Varma et al., “T Cell Receptor-Proximal Signals are Sustained in Peripheral Microclusters and Terminated in the Central Supramolecular Activation Cluster,” Immunity 25:117-127 (2006), which is hereby incorporated by reference in its entirety). Briefly, cells interacted with the bilayers for 8 minutes at 37° C. and were fixed with 2% PFA; permeabilized with 0.05% Triton X-100; blocked and stained with rabbit polyclonal antibodies to Dlgh1 (H-60; sc-25661), PKC-θ (sc-212), or Carmal (Card 11, C-12) from Santa Cruz Biotech for 20 minutes; and then incubated with fluorescently tagged goat anti-rabbit Fab2 (Invitrogen). Controls included the use of nonimmune species-matched IgG. Measurement of signaling was done as described (Zanin-Zhorov et al., “Protein Kinase C-Theta Mediates Negative Feedback on Regulatory T Cell Function,” Science 328:372-376 (2010), which is hereby incorporated by reference in its entirety). Confocal microscopy was carried out on a Zeiss LSM 510 Meta imaging system (63×1.4 NA; Zeiss) using appropriate factory-set filters and dichroics for different fluorophores as described (Zanin-Zhorov et al., “Protein Kinase C-Theta Mediates Negative Feedback on Regulatory T Cell Function,” Science 328:372-376 (2010), which is hereby incorporated by reference in its entirety).

In Vitro Suppression Assays—CD4⁺CD25⁺ T cells were treated or not, washed, and added at a ratio of 1:3 (1.25×10⁵: 5×10⁵) to CD4⁺CD25⁻ T cells at final concentration of 2×10⁶ per mL (cytokine secretion) or 2×10⁵ per mL (proliferation). The cells were co-cultured on anti-CD3 mAb (5 μg/mL) pre-coated 24-well plates for 24-48 hours (cytokine secretion) or 96 hours (proliferation). Human TNF-α (210-TA) was purchased from R&D Systems and added to co-cultures where indicated. The PKC-θ inhibitor, compound 20, was provided by Boehringer-Ingelheim Pharmaceuticals and dissolved in DMSO (Cywin et al., “Discovery of Potent and Selective PKC-Theta Inhibitors,” Bioorg. Med. Chem. Lett. 17:225-230 (2007), which is hereby incorporated by reference in its entirety). T cells were pretreated for 30 minutes at a concentration of 1 μM at 37° C. and washed three times. Cytokine secretion was determined by ELISA as described (Zanin-Zhorov et al., “Protein Kinase C-Theta Mediates Negative Feedback on Regulatory T Cell Function,” Science 328:372-376 (2010), which is hereby incorporated by reference in its entirety), using human IFN-γ Cytoset (Biosource) and IL-17 and -4 (Invitrogen). Proliferation was assessed by carboxyfluorescein diacetate succinimidyl ester (CFSE) dilution as described (Tran et al., “Analysis of Adhesion Molecules, Target Cells, and Role of IL-2 in Human FOXP3+ Regulatory T Cell Suppressor Function,” J. Immunol. 182:2929-2938 (2009), which is hereby incorporated by reference in its entirety).

Flow Cytometry—

Indicated populations of T cells were stained (30 minutes, 4° C.) with PE-labeled anti-CD25 (Miltenyi Biotec) and FITC-labeled anti-CD127 (eBioscience) antibodies and washed with PBS (containing 0.05% BSA and 0.05% sodium azide). For intracellular staining, cells were fixed and permeabilized with Fixation/Permeabilization buffer set (00-5523; eBioscience), washed, and stained (30 minutes, 4° C.) with primary antibodies [PE-labeled Foxp3 (PCH101) or Dlgh1]. Then, the cells were incubated (30 minutes, 4° C.) with FITC-conjugated secondary antibodies (Jackson ImmunoResearch). Samples were analyzed in a FACSCalibur machine (BD).

RNAi—

siRNA duplexes (siRNAs) were synthesized and purified by Qiagen as described (Srivastava et al., “Engagement of Protein Kinase C-Theta in Interferon Signaling in T-Cells,” J. Biol. Chem. 279:29911-29920 (2004), which is hereby incorporated by reference in its entirety). A mixture of four Dlgh1-specific siRNAs was used [catalog nos. SI00059584 (Dlgh1-1), SI02632518 (Dlgh1-7), SI03046099 (Dlgh1-8), and SI03102799 (Dlgh1-9)]. Control siRNA was purchased from Qiagen (1027281). Transfections of freshly purified T cells were performed by using the human T-cell Nucleofector kit (Amaxa Biosystems, Lonza) as described (Zanin-Zhorov et al., “Protein Kinase C-Theta Mediates Negative Feedback on Regulatory T Cell Function,” Science 328:372-376 (2010), which is hereby incorporated by reference in its entirety).

Western Blot and Immunoprecipitation—

Cells were lysed in radioimmunoprecipitation assay buffer (pH 8) supplemented with protease and phosphatase inhibitors. After 20 minutes of centrifugation at 10,000×g at 4° C., Dlgh1 and PTEN were immunoprecipitated by incubation for 1 hour at 4° C. with 2 μg of anti-Dlgh1 antibody (610875; BD Transduction Laboratories), antiPTEN (B-1; sc-133197; Santa Cruz Biotech), or normal mouse IgG (sc-2025; Santa Cruz Biotech) followed by overnight incubation with protein A/G PLUS-Agarose beads (sc-2003; Santa Cruz Biotech). The immunoprecipitates were washed five times with cold PBS, loaded on an SDS/PAGE gel, and transferred to nitrocellulose membrane. The membranes were blocked, probed with the specific antibodies overnight, washed, and stained with secondary antibodies from Li-Cor. Immunoreactive protein bands were visualized by using an Odyssey Infrared Imaging system. Anti-alpha actin antibodies were used as loading controls.

NFATc1 and NF-κB/p50 Activation Assays—

Cells were activated on anti-CD3 mAb (5 μg/mL) and lysed, and NFATc1-activation and p50-specific binding to NF-κB consensus sequence were tested by TransFactor NFATc1 Chemiluninescent Kit and TransFactor NF-κB p50 Colorimetric Kit, respectively (nos. 631916 and 631955; Clontech Laboratories), according to manufacturer's instructions.

Statistics—

P values were determined by Mann-Whitney or two-tailed t test by using the GraphPad Prism software.

### Example 2

### Dlgh1 is Strongly Recruited to the Treg IS and Plays a Unique Role in Treg Signaling

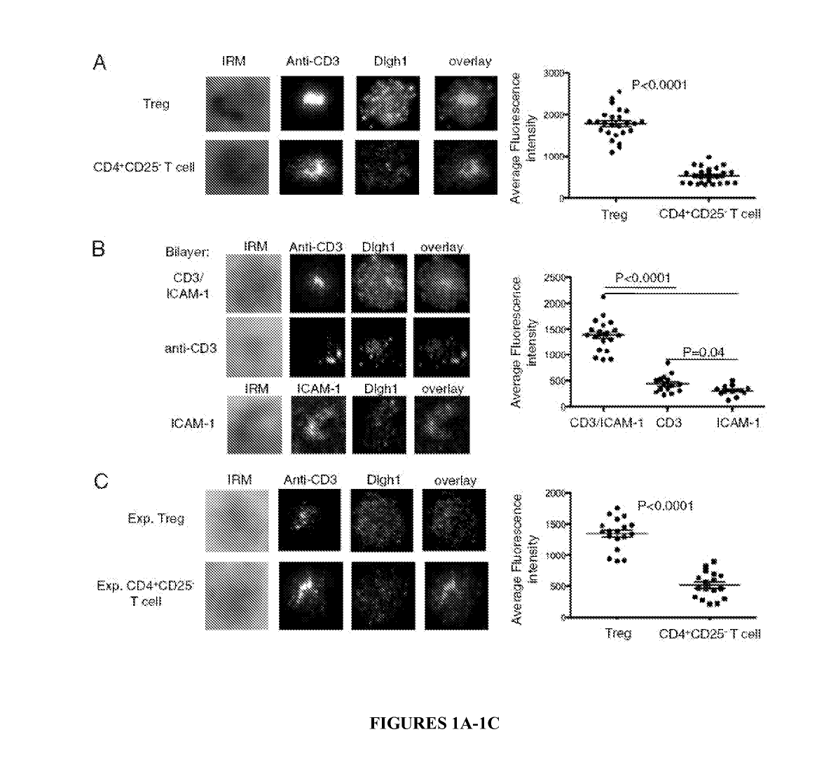

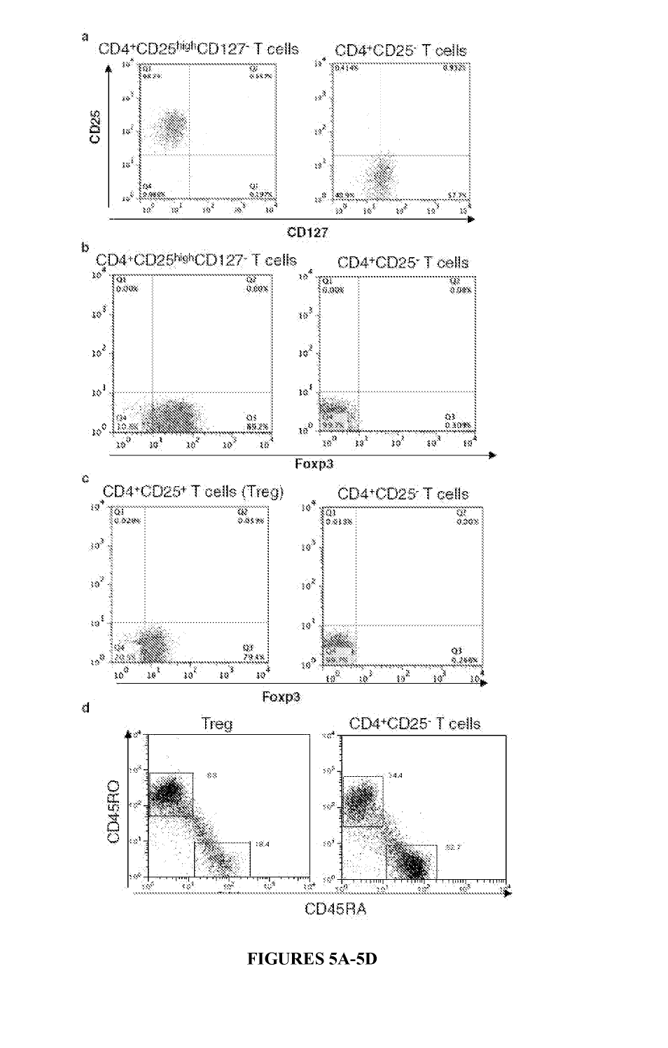

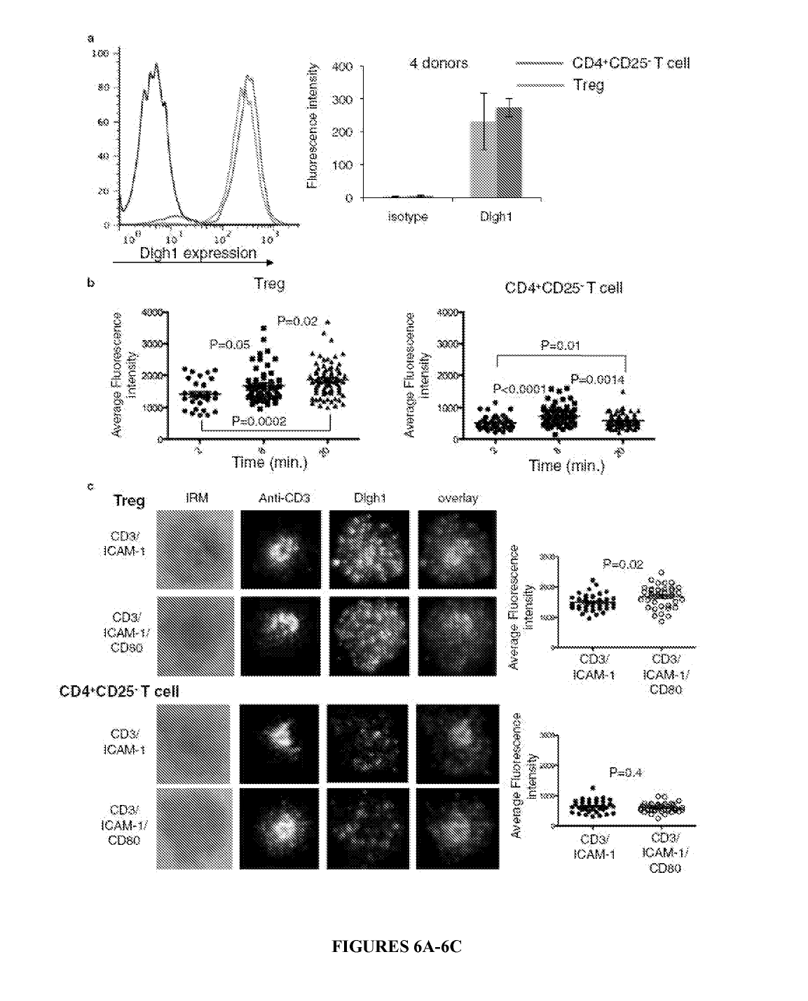

To investigate the role of Dlgh1 in activation of in vitro Treg function, it was first determined whether Dlgh1 is recruited to the IS. Human CD4⁺CD25^{high}CD127^{low} cells were isolated by flow cytometry (FIG. 5A) and 90% of these were Foxp3⁺ (FIG. 5B) or by positive selection by MACS (75%-80% are Foxp3⁺; FIG. 5C). The Treg are 80% CD45RO⁺, suggesting they have been previously activated by antigen in vivo (FIG. 5D) (Miyara et al., “Functional Delineation and Differentiation Dynamics of Human CD4⁺ T Cells Expressing the FoxP3 Transcription Factor,” Immunity 39(6):899-911 (2009); Valmori et al., “A Peripheral Circulating Compartment of Natural Naïve CD4 Tregs,” J. Clin. Invest. 115(7):1953-1962 (2005), both of which are hereby incorporated by reference in their entirety). In order to compare IS of TregS and CD4⁺CD25⁻ T cells under identical conditions, cells on planar bilayers containing mobile fluorescently labeled ICAM-1 and the stimulatory anti-CD3 antibodies were incubated for 8 minutes. The cells were then fixed, permeabilized, stained with affinity-purified antibodies to Dlgh1, and imaged by total internal reflection fluorescence microscopy (TIRFM). TIRFM only detects fluorescence within 200 nm of the interface between the T cells and the planar bilayers. There was four-fold increase in the intensity of anti-Dlgh1 staining in FACS-sorted (FIG. 1A), as well as MACS bead-purified (FIG. 1B), Tregs compared with CD4⁺CD25⁻ T cells under the same conditions, although there were no differences in total intracellular levels of the protein between two cell populations (FIG. 1A; FIG. 6A).

Kinetic analysis demonstrated that the recruitment of Dlgh1 to IS in Tregs was slightly increased between 8 and 20 minutes (FIG. 6B), whereas Dlgh1 accumulation at IS in CD4⁺CD25⁻ T cells was transient and peaked at 8 minutes (FIG. 6B). Introduction of the costimulatory signal CD80 into bilayers slightly increased the Dlgh1 recruitment to IS in Tregs and had no significant effect in CD4⁺CD25⁻ T cells (FIG. 6C). Incubation of Treg on bilayers containing anti-CD3 antibodies or ICAM-1 alone revealed that both TCR— and integrin-mediated signals respectively are required for maximum recruitment of Dlgh1 (FIG. 1B). A critical role of LFA-1/ICAM-1 interaction is consistent with functional studies demonstrating the importance of LFA-1 for Treg function (Tran et al., “Analysis of Adhesion Molecules, Target Cells, and Role of IL-2 in Human FOXP3+Regulatory T Cell Suppressor Function,” J. Immunol. 182(5):2929-2938 (2009), which is hereby incorporated by reference in its entirety). By using ex vivo expanded human umbilical cord blood derived Tregs (Hippen et al., “Umbilical Cord Blood Regulatory T-Cell Expansion and Functional Effects of Tumor Necrosis Factor Receptor Family Members OX40 and 4-1BB Expressed on Artificial Antigen-Presenting Cells,” Blood 112(7):2847-2857 (2008), which is hereby incorporated by reference in its entirety), the same pattern of increased enrichment of Dlgh1 at the IS compared to CD4⁺CD25⁻ T cells expanded under the same conditions was found (FIG. 1C). Thus, Dlgh1 is strongly recruited to the Treg IS and is well positioned to play a unique role in Treg signaling.

### Example 3

### Dlgh1 Enrichment at IS Correlates with Treg Suppressive Function

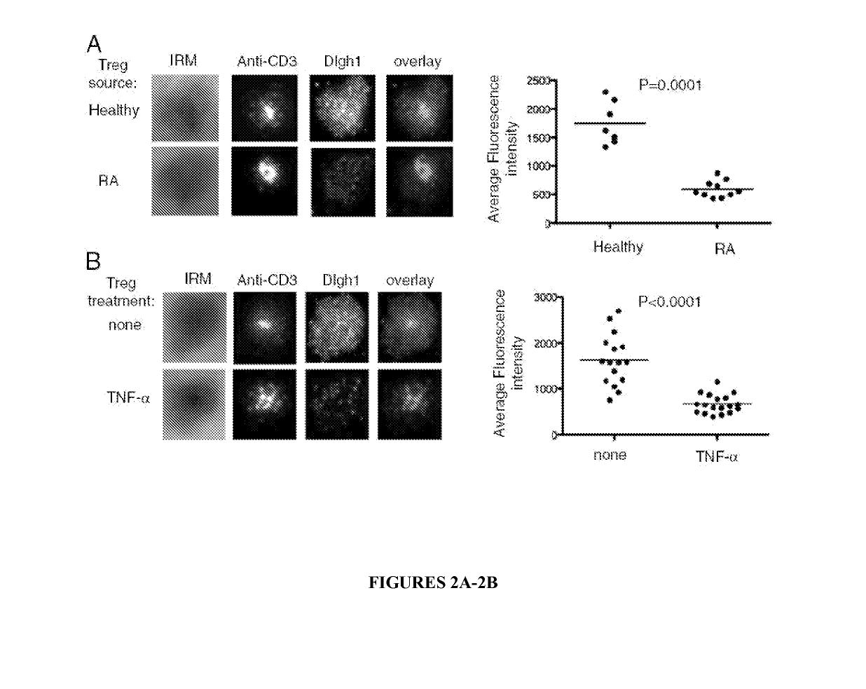

The pro-inflammatory mediator TNF-α inhibits suppressive activity and down-regulates Foxp3 in human Treg (Valencia et al., “TNF Downmodulates the Function of Human CD4+CD25hi T-Regulatory Cells,” Blood 108(1):253-261 (2006); Zanin-Zhorov et al., “Protein Kinase C-Theta Mediates Negative Feedback on Regulatory T Cell Function,” Science 328(5976):372-376 (2010), both of which are hereby incorporated by reference in their entirety). To investigate whether Dlgh1 recruitment to IS correlates with suppressive function of Treg, Treg was incubated with TNF-α overnight and then analyzed Dlgh1 recruitment to the IS as described above. It was found that treatment with TNF-α significantly reduced levels of Dlgh1 at IS in Treg (FIG. 2A). Patients with rheumatoid arthritis (“RA”) have normal numbers of Tregs, but their suppressive function is decreased based on in vitro assays (Valencia et al., “TNF Downmodulates the Function of Human CD4+CD25hi T-Regulatory Cells,” Blood 108(1):253-261 (2006); Zanin-Zhorov et al., “Protein Kinase C-Theta Mediates Negative Feedback on Regulatory T Cell Function,” Science 328(5976):372-376 (2010); Ehrenstein et al., “Compromised Function of Regulatory T Cells in Rheumatoid Arthritis and Reversal by Anti-TNFalpha Therapy,” J. Exp. Med. 200(3):277-285 (2004), all of which are hereby incorporated by reference in their entirety). To investigate whether Dlgh1 recruitment to IS has functional consequences in Tregs, imaging and flow cytometric analysis of samples from 10 RA patients with moderated to severe systemic inflammation was performed [Disease Activity Score (DAS) between 4.32 and 6.63] and not treated with anti-TNF-α therapy (FIG. 7A) and found that, whereas total levels of Dlgh1 were not significantly different between freshly purified healthy and RA Tregs, the levels of Dlgh1 recruited to IS were significantly lower in RA Tregs compared with healthy controls (FIG. 7B and FIG. 2A). It has been reported that the proinflammatory mediator TNF-α inhibits Treg-suppressive activity in vitro, down-regulates Foxp3, and is required for defective Treg activity in RA patients (Valencia et al., “TNF Downmodulates the Function of Human CD4+CD25hi T-Regulatory Cells,” Blood 108:253-261 (2006), Zanin-Zhorov et al., “Protein Kinase C-Theta Mediates Negative Feedback on Regulatory T Cell Function,” Science 328:372-376 (2010), both of which are hereby incorporated by reference in their entirety)). To evaluate the effects of TNF-α, Tregs purified from healthy donors were incubated with 50 ng/mL TNF-α over-night and then Dlgh1 recruitment to the IS was analyzed as described above. It was found that treatment with TNF-α significantly reduced levels of Dlgh1 at IS in Tregs (FIG. 2B). Thus, Dlgh1 enrichment at the IS strongly correlates with Treg-suppressive function.

### Example 4

### Role of Dlgh1 in Treg Suppressive Function is Conserved

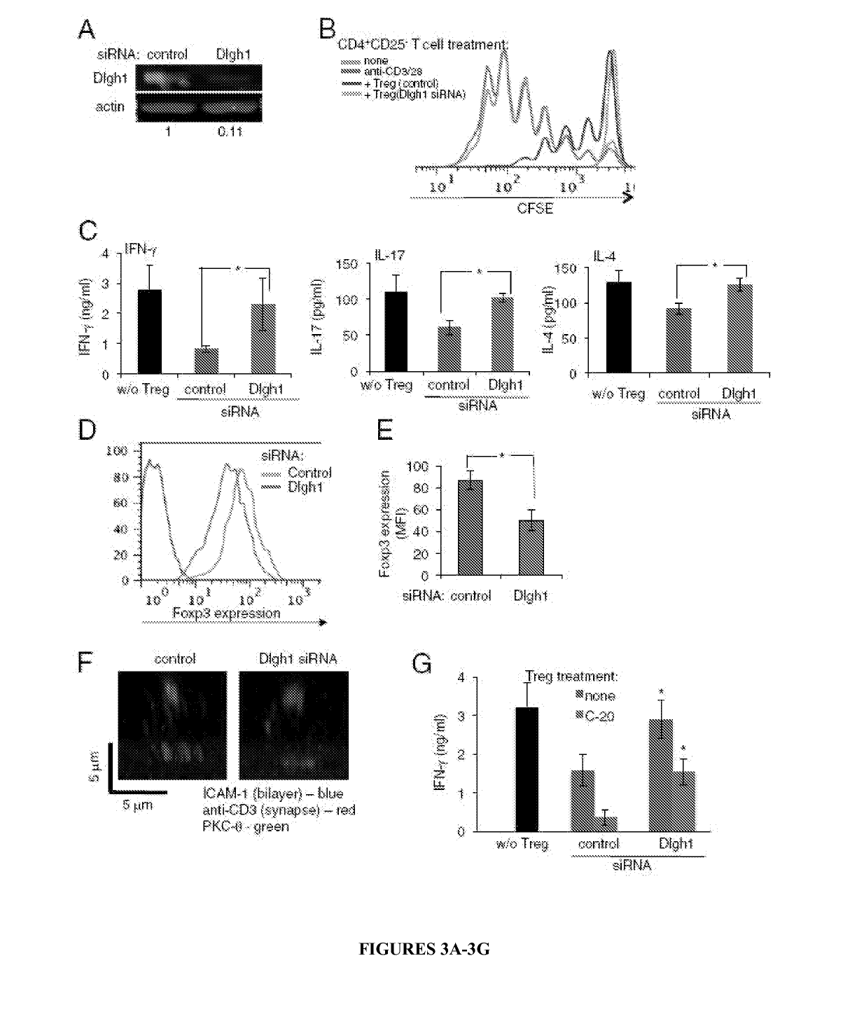

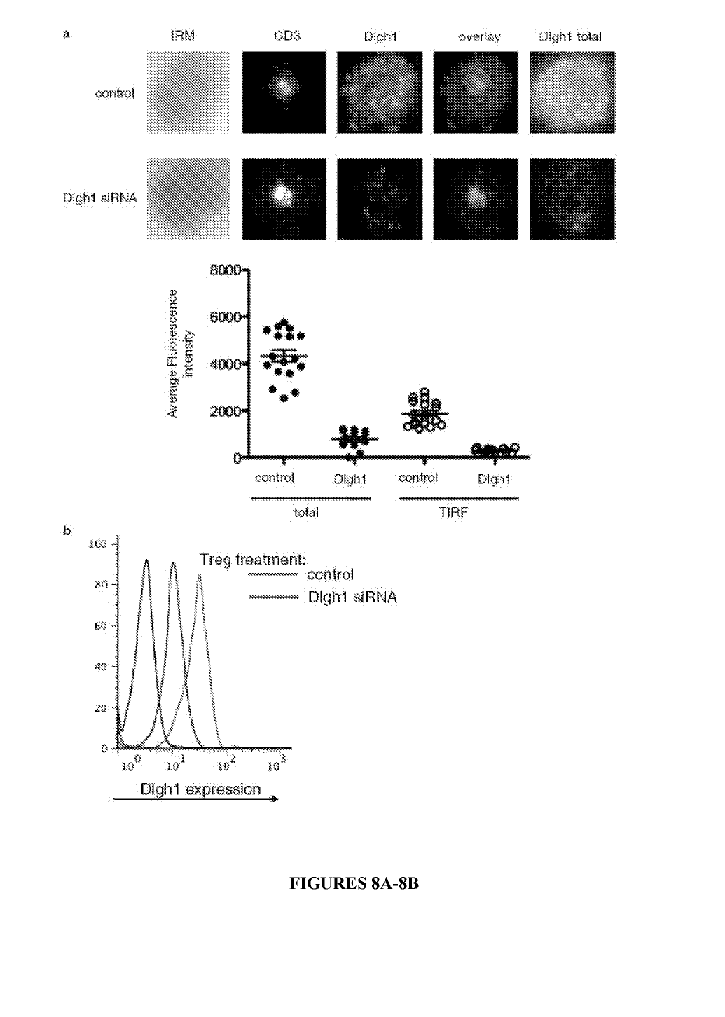

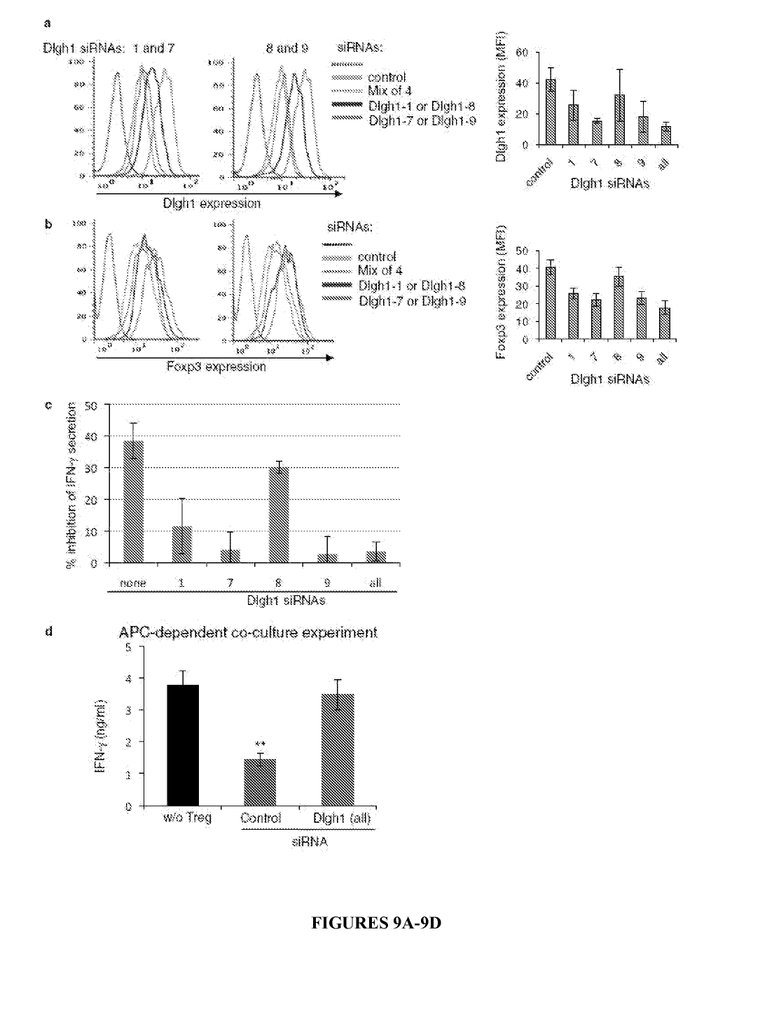

To investigate whether Dlgh1 is important for Treg suppressive function, Dlgh1 gene expression was specifically silenced using RNA interference (RNAi). Treatment with a mixture of four specific siRNAs for Dlgh1 resulted in an 89% reduction of Dlgh1 expression in freshly purified Tregs (FIG. 3A and FIG. 8A-8B). This reduction of Dlgh1 in human Tregs significantly impaired the ability to inhibit CD4⁺CD25⁻ T cell proliferation (FIG. 3B), as well as secretion of IFN-γ, IL-17, and IL-4 (FIG. 3C). Notably, reduction of Dlgh1 by siRNA resulted in a marked decrease of Foxp3 expression (FIGS. 3D and 3E). To exclude the possibility of off-target effects, the impact of each siRNA duplex was tested separately and it was found that the efficacy of those specific siRNAs to knock down Dlgh1 expression correlated with the ability to down-regulate Foxp3 expression and the suppressive function of Tregs (FIGS. 9A-9C). Because each siRNA duplex would be expected to target distinct off-target mRNAs, the correlation across the four duplexes supports the conclusion that Dlgh1 is required for Treg FoxP3 expression and suppression of IFN-γ production by CD4⁺CD25⁻ T cells. The same inhibition of Treg function by Dlgh1 suppression was observed in the antigen-presenting cell-dependent Treg assay (FIG. 9D).

### Example 5

### Dlgh1 Coordinates Two Signaling Pathways in Treg that Generate an Atypical TCR Signaling Pattern in Treg where NFATc1 Activation is High and Akt and NF-κB Activation are Low

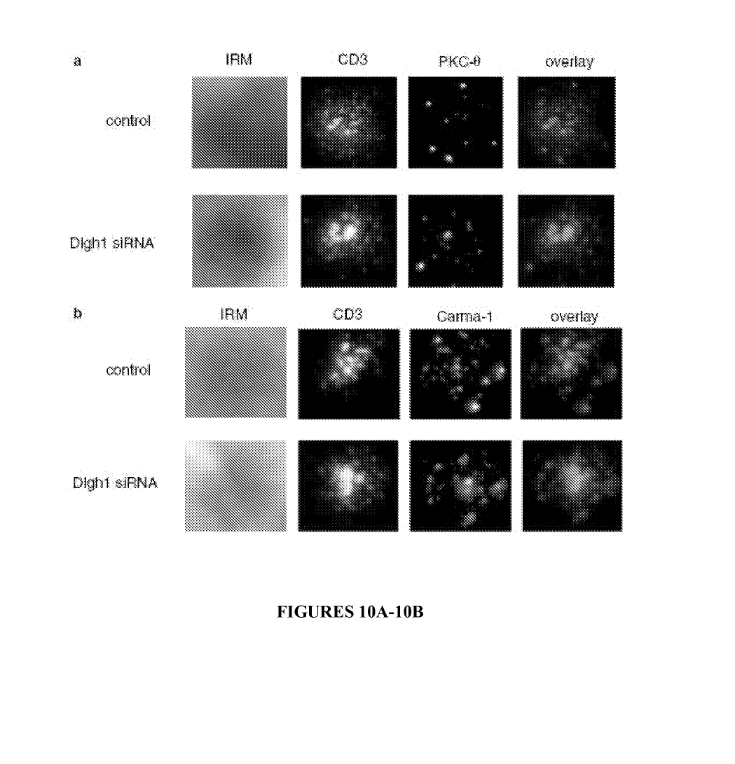

Finally, it was investigated whether there is crosstalk between Dlgh1 and the negative feedback pathway involving PKC-θ (Zanin-Zhorov et al., “Protein Kinase C-Theta Mediates Negative Feedback on Regulatory T Cell Function,” Science 328(5976):372-376 (2010), which is hereby incorporated by reference in its entirety). This is a particularly relevant question because the key PKC-θ substrate Carma-1 is also a member of the MAGUK family (Blonska et al., “CARMAl-Mediated NF-kappaB and JNK Activation in Lymphocytes,” Immunol. Reviews 228(1):199-211 (2009); Rebeaud et al., “Dlgh1 and Carmal MAGUK Proteins Contribute to Signal Specificity Downstream of TCR Activation,” Trends Immunol. 28(5):196-200 (2007), both of which are hereby incorporated by reference in their entirety). Imaging analysis of Dlgh1 suppressed human Tregs revealed that Dlgh1 is not required for PKC-θ localization in the distal pole upon formation of IS in Treg (FIGS. 10A, 10B). Moreover, treatment of Dlgh1 silenced Treg with specific PKC-θ inhibitor C20, only partially restored Treg suppressive function (FIGS. 3F and 3G). These findings suggest that PKC-θ-mediated negative and Dlgh1-mediated positive pathways regulate Treg function independently and that PKC-θ inhibition cannot fully compensate for the loss of Dlgh1. Thus, defects in Dlgh1 recruitment to the IS of Treg from patients with RA is one of the PKC-θ independent mechanisms of Treg dysfunction in RA.

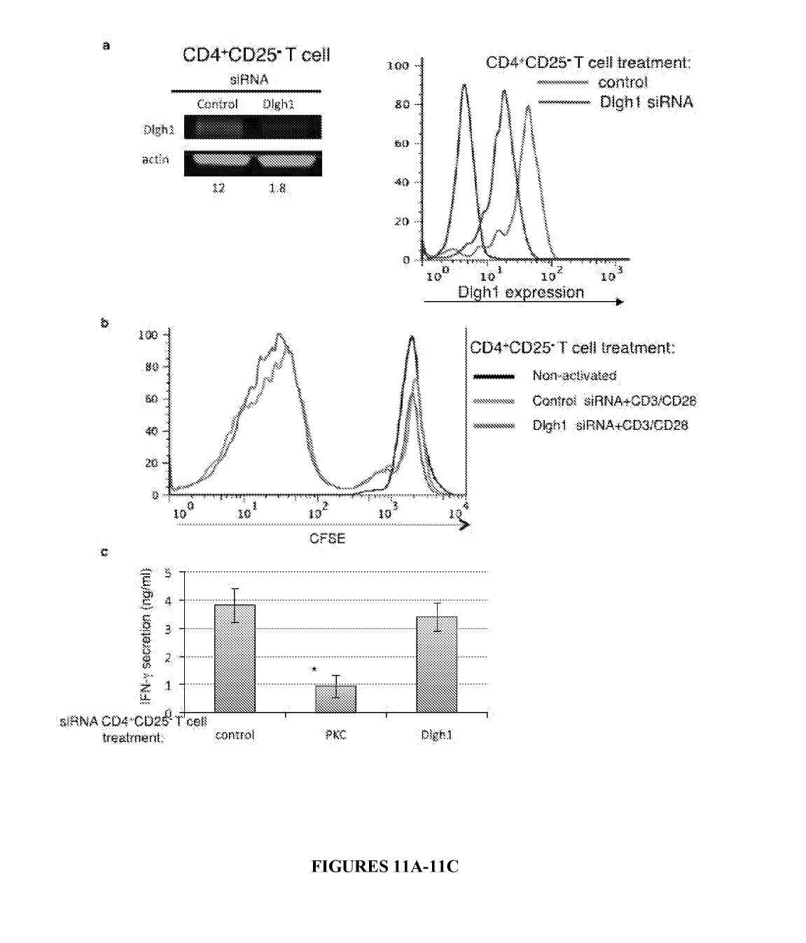

Dlgh1 has been reported to have a positive or negative role in regulation of Teff function (Round et al., “Dlgh1 Coordinates Actin Polymerization, Synaptic T Cell Receptor and Lipid Raft Aggregation, and Effector Function in T Cells,” J. Exp. Med. 201(3):419-430 (2005); Stephenson et al., “DLGH1 is a Negative Regulator of T-Lymphocyte Proliferation,” Mol. Cell. Biol. 27(21):7574-7581 (2007), both of which are hereby incorporated by reference in their entirety). Again, siRNA was used to investigate the effect of Dlgh1 down-reguation (FIG. 11A) on cytokine secretion and proliferation of human CD4⁺ CD25⁻ T cells. It was found that silencing of Dlgh1 did not affect the ability of CD4⁺ CD25⁻ T cells to proliferate (FIG. 11B) or secrete IFN-γ (FIG. 11C) in response to TCR stimulation. Thus, Dlgh1 is not required for TCR-induced CD4⁺ CD25⁻ T cell function under the conditions applied here.

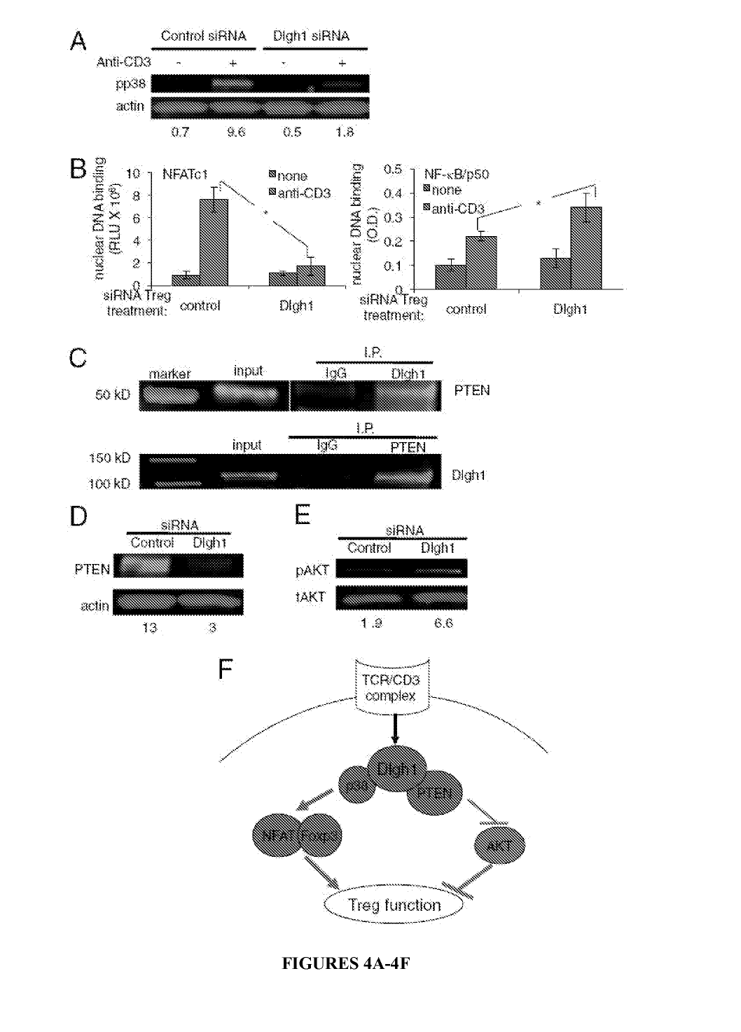

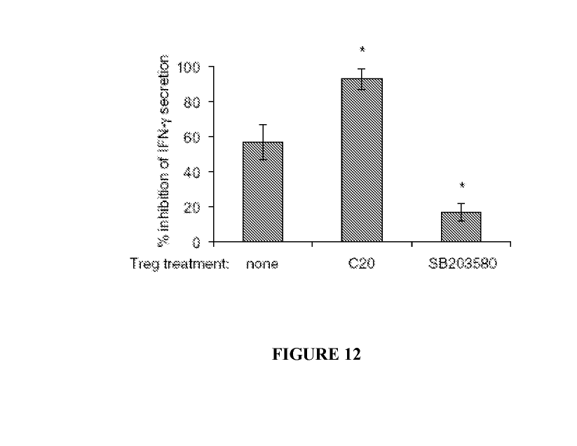

NFATc1 activation is required for Treg-suppressive function (Wu et al., “FOXP3 Controls Regulatory T Cell Function Through Cooperation with NFAT,” Cell 126(2):375-387 (2006), which is hereby incorporated by reference in its entirety). These studies were initiated with the model that Dlgh1 mediates NFATc1 through p38, as proposed by Micelli and colleagues (Round et al., “Scaffold Protein Dlgh1 Coordinates Alternative p38 Kinase Activation, Directing T Cell Receptor Signals Toward NFAT but not NF-KappaB Transcription Factors,” Nat. Immunol. 8:154-161 (2007), which is hereby incorporated by reference in its entirety). This model predicts that inhibition of p38 or silencing Dlgh1 would decrease p38 phosphorylation at Thr-180/Tyr-182 and decrease NFATc1 activation. Pre-treatment of Treg with specific p38 inhibitor, SB203580, significantly down-regulated their ability to suppress IFN-γ secretion from CD4⁺ CD25⁻ T cells (FIG. 12). Moreover, silencing of Dlgh1 in Tregs inhibited p38 phosphorylation (FIG. 4A) and NFATc1 activation (FIG. 4B) in response to TCR stimulation. Thus, Dlgh1 contributes to activation of p38 and NFATc1 in Tregs.

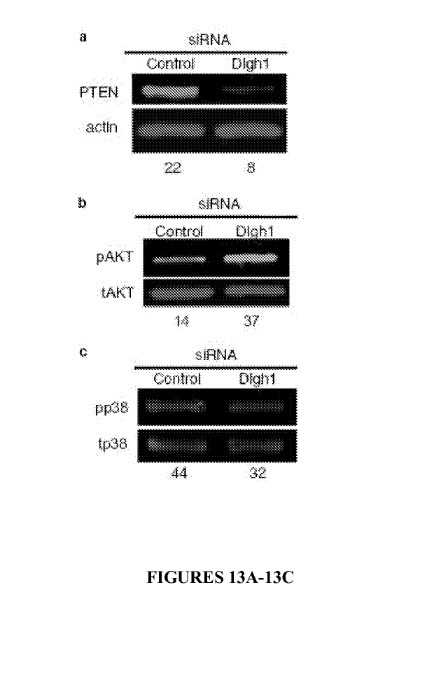

Optimal Treg function requires reduced Akt activation compared with Teff (Crellin et al., “Altered Activation of AKT is Required for the Suppressive Function of Human CD4+CD25+ T Regulatory Cells,” Blood 109(5):2014-2022 (2007); Liu et al., “S1P₁-mTOR Axis Directs the Reciprocal Differentiation of T(H)1 and T(Reg) Cells,” Nat. Immunol. 11(11):1047-1056 (2010), which are hereby incorporated by reference in their entirety). Akt membrane recruitment and activation depends upon phosphatidylinositol-3,4,5 triphosphate, which is destroyed by PTEN (Salmena et al., “Tenets of PTEN Tumor Suppression,” Cell 133(3):403-414 (2008), which is hereby incorporated by reference in its entirety). Dlgh1 interacts with PTEN through a PDZ domain, stabilizes and recruits it to the membrane where PTEN is active (Cotter et al., “Dlg1-PTEN Interaction Regulates Myelin Thickness to Prevent Damaging Peripheral Nerve Overmyelination,” Science 328(5984):1415-1418 (2010); Adey et al., “Threonine Phosphorylation of the MMAC1/PTEN PDZ Binding Domain Both Inhibits and Stimulates PDZ Binding,” Cancer Res. 60(1):35-37 (2000); Valiente et al., “Binding of PTEN to Specific PDZ Domains Contributes to PTEN Protein Stability and Phosphorylation by Microtubule-Associated Serine/Threonine Kinases,” J. Biol. Chem. 280(32):28936-28943 (2005), all of which are hereby incorporated by reference in their entirety). Studies of Dlgh1 were initiated based on the model that Dlgh1 could stabilize and recruit PTEN to suppress Akt activation. Indeed, coimmunoprecipitation analysis confirmed Dlgh1/PTEN interaction in Tregs (FIG. 4C). Furthermore, by using specific antibody against PTEN, it was found that Dlgh1 silencing reduced PTEN levels by 75% in both Tregs and CD4⁺ CD25⁻ T cells (FIG. 4D and FIG. 13A). Moreover, Dlgh1 silencing in Tregs and CD4⁺ CD25⁻ increased levels of Akt phosphorylation on Ser-473, a signature of membrane recruitment and activation, in response to TCR stimulation (FIG. 4E and FIG. 13B). Dlgh1 silencing also resulted in increased NF-κB activation in Treg (FIG. 4B), which may be directly related to increased Akt activity since it was demonstrated supra that the PKC-θ pathway is not regulated by Dlgh1.

Thus, Dlgh1 coordinates two critical signaling pathways in Treg that generate an atypical TCR signaling pattern in Treg in which NFATc1 activation is high and Akt and NF-κB activation are low. These ends are achieved through the ability of the Dlgh1 to mediate p38-dependent activation of NFAT and inhibit Akt and NF-κB signaling pathways through PTEN stabilization in Tregs (FIG. 4F). It is notable that two members of the MAGUK family, Carmal and Dlgh1, provide scaffolds for apparently independent, but opposing signaling pathways in Treg, both of which are dysregulated in rheumatoid arthritis. These opposing scaffolds offer a new array of targets for up or down regulation of Treg function in different therapeutic contexts.

### Example 6

### Dlgh1 Mediates Key Activating Signal Downstream of the TCRs that Operates in Opposition to the PKC-0/Carmal-mediated Negative Feedback Pathway in Tregs

TCR signals activate the opposing functional programs of Teffs and Tregs (Sakaguchi et al., “Regulatory T Cells and Immune Tolerance,” Cell 133:775-787 (2008), which is hereby incorporated by reference in its entirety). TCR signaling pathways defined first in Teffs have been shown to behave differently in Tregs (Ohkura et al., “Regulatory T Cells: Roles of T Cell Receptor for Their Development and Function,” Semin. Immunopathol. 32:95-106 (2010), which is hereby incorporated by reference in its entirety). It was recently discovered that PKC-θ and Carmal, major signaling components in the NF-κB activating pathway, mediate negative feedback signaling for Treg function (Zanin-Zhorov et al., “Protein Kinase C-Theta Mediates Negative Feedback on Regulatory T Cell Function,” Science 328:372-376 (2010), which is hereby incorporated by reference in its entirety). Similarly, Akt signaling promotes Teff activation, but levels of activation observed in Teff are inhibitory to Treg function (Crellin et al., “Altered Activation of AKT is Required for the Suppressive Function of Human CD4⁺CD25⁺ T Regulatory Cells,” Blood 109:2014-2022 (2007), which is hereby incorporated by reference in its entirety)). Finally, ZAP-70 kinase activity is essential for Teff signaling, but not for Treg activation, although ZAP-70 plays a scaffolding role for LFA-1 activation in Treg (Au-Yeung et al., “A Genetically Selective Inhibitor Demonstrates a Function for the Kinase Zap70 in Regulatory T Cells Independent of its Catalytic Activity,” Nat. Immunol. 11:1085-1092 (2010), which is hereby incorporated by reference in its entirety). Here, it is demonstrated that Dlgh1 scaffolds two critical signaling pathways in Tregs that generate a distinct TCR signaling network, in which NFATc1 activation is high and Akt and NF-κB activation are low. These outcomes are associated with the ability of the Dlgh1 scaffold to mediate p38 activation and PTEN stabilization.

Dlgh1 selectively activates p38 in antigen-experienced T cells, whereas naïve T cells preferentially induce ERK phosphorylation in response to TCR activation (Adachi et al., “T-Cell Receptor Ligation Induces Distinct Signaling Pathways in Naive vs. Antigen-Experienced T Cells,” Proc. Natl. Acad. Sci. USA 108:1549-1554 (2011), which is hereby incorporated by reference in its entirety). Human peripheral blood Tregs are highly skewed toward antigen-experienced cells (Valmori et al., “A Peripheral Circulating Compartment of Natural Naive CD4 Tregs,” J. Clin. Invest. 115:1953-1962 (2005), which is hereby incorporated by reference in its entirety). Naïve Tregs are a minor population in humans, and it is not clear whether this population also uses Dlgh1, because the contribution of these cells may be obscured by the memory cells. The data of the present invention is fully consistent with earlier models showing that Dlgh1 forms a scaffold for ZAP-70 and Lck to recruit and activate p38 (Round et al., “Scaffold Protein Dlgh1 Coordinates Alternative p38 Kinase Activation, Directing T Cell Receptor Signals Toward NFAT but Not NF-kappaB Transcription Factors,” Nat. Immunol. 8:154-161 (2007), which is hereby incorporated by reference in its entirety). ZAP-70 catalytic activity is not needed for mouse Treg function (Au-Yeung et al., “A Genetically Selective Inhibitor Demonstrates a Function for the Kinase Zap70 in Regulatory T Cells Independent of its Catalytic Activity,” Nat. Immunol. 11:1085-1092 (2010), which is hereby incorporated by reference in its entirety), but further work is needed to determine whether ZAP-70 catalytic activity is also dispensable for Dlgh1-dependent human Treg activation.

The role of PTEN and phosphatidylinositol-3-kinase (PI3K) in controlling Treg function is not fully understood. PTEN catalyzes the reverse reaction of PI3K and thereby negatively regulates the activation of downstream signaling pathways (Leslie et al., “PTEN: The Down Side of PI 3-Kinase Signalling,” Cell Signal 14:285-295 (2002), which is hereby incorporated by reference in its entirety). In mice, PTEN levels are relatively higher in Tregs compared with Teffs where PTEN is down-regulated in response to TCR stimulation (Bensinger et al., “Distinct IL-2 Receptor Signaling Pattern in CD4⁺CD25⁺ Regulatory T Cells,” J. Immunol. 172:5287-5296 (2004), which is hereby incorporated by reference in its entirety). Although targeted deletion of PTEN up-regulates the IL-2-mediated expansion of Tregs without affecting their development and suppressive function (Walsh et al., “PTEN Inhibits IL-2 Receptor-Mediated Expansion of CD4⁺ CD25⁺ Tregs,” J. Clin. Invest. 116:2521-2531 (2006), which is hereby incorporated by reference in its entirety), constitutive activation of PI3K/AKT/mTOR signaling pathway antagonized Foxp3 induction (Sauer et al., “T Cell Receptor Signaling Controls Foxp3 Expression Via PI3K, Akt, and mTOR,” Proc. Natl. Acad. Sci. USA 105:7797-7802 (2008), which is hereby incorporated by reference in its entirety). Moreover, sphingosine 1-phosphate receptor 1 inhibits Treg differentiation through mTOR signaling, a target of the Akt pathway (Liu et al., “The Receptor S1P1 Overrides Regulatory T Cell-Mediated Immune Suppression Through Akt-mTOR,” Nat. Immunol. 10:769-777 (2009), which is hereby incorporated by reference in its entirety). Some of the discrepancy between the role of PTEN and Akt in Tregs might be explained by evidence that PTEN possesses functions that are independent of its ability to specifically suppress the PI3K pathway (Blanco-Aparicio et al., “PTEN, More Than the AKT Pathway,” Carcinogenesis 28:1379-1386 (2007), which is hereby incorporated by reference in its entirety). In humans, Tregs demonstrate reduced Akt activation, and restoration of Akt activity to levels in Teffs inhibits Treg function in vitro (Crellin et al., “Altered Activation of AKT is Required for the Suppressive Function of Human CD4⁺CD25⁺ T Regulatory Cells,” Blood 109:2014-2022 (2007), which is hereby incorporated by reference in its entirety) suggesting that the PI3K/Akt signaling pathway negatively regulates human Tregs. Consistent with this finding, the results of the present invention demonstrate that Dlgh1-mediated decreases of intracellular levels of PTEN lead to increased TCR-induced Akt activation and significant down-regulation of the suppressive Treg function. Studies from other cellular systems have demonstrated that Dlgh1 binds to PTEN through its C-terminal PDZ domain binding motif and prevents PTEN degradation (Cotter et al., “Dlg1-PTEN Interaction Regulates Myelin Thickness to Prevent Damaging Peripheral Nerve Overmyelination,” Science 328:1415-1418 (2010); Valiente et al., “Binding of PTEN to Specific PDZ Domains Contributes to PTEN Protein Stability and Phosphorylation by Microtubule-Associated Serine/Threonine Kinases,” J. Biol. Chem. 280:28936-28943 (2005), which is hereby incorporated by reference in its entirety). Further biochemical characterization is required to define the precise molecular mechanism of Dlgh1 involvement in PTEN stabilization and investigate the possible role of this protein in regulation of Foxp3 levels in T cells.

Dlgh1, also known at SAP97, plays a central role in the organization of postsynaptic densities and epithelial apical junctions in many tissues (Funke et al., “Membrane-Associated Guanylate Kinases Regulate Adhesion and Plasticity at Cell Junctions,” Annu. Rev. Biochem. 74:219-245 (2005); Valtschanoff et al., “SAP97 Concentrates at the Postsynaptic Density in Cerebral Cortex,” Eur. J. Neurosci. 12:3605-3614 (2000); Yamanaka et al., “Role of Lgl/D1g/Scribble in the Regulation of Epithelial Junction, Polarity and Growth,” Front Biosci. 13:6693-6707 (2008), which are hereby incorporated by reference in their entirety). Dlgh1 appears to be have assumed a specific role in activation of Treg function at the IS with greater recruitment to the Treg IS, and, in the hands of researchers of the present invention, its depletion had no impact on TCR-induced cytokine production or proliferation of human CD4⁺CD25⁻ T cells (FIG. 11A-11C), in contrast to previous reports with activated CD8⁺ mouse T cells (Round et al., “Scaffold Protein Dlgh1 Coordinates Alternative p38 Kinase Activation, Directing T Cell Receptor Signals Toward NFAT but Not NF-kappaB Transcription Factors,” Nat. Immunol. 8:154-161 (2007), which is hereby incorporated by reference in its entirety). One possibility is that because the majority of human CD4⁺CD25⁻ T cells are naïve (FIG. 5D), they rely on ERK-mediated signaling rather than on Dlgh1/p38 signaling pathways (Adachi et al., “T-Cell Receptor Ligation Induces Distinct Signaling Pathways in Naive vs. Antigen-Experienced T Cells,” Proc. Natl. Acad. Sci. USA 108:1549-1554 (2011), which is hereby incorporated by reference in its entirety). Indeed, Dlgh1 silencing in CD4⁺CD25⁻ T cells resulted in only 30% down-regulation in TCR-induced p38 phosphorylation compared with 80% inhibition in Tregs (FIG. 13C). In addition, the fact that Dlgh1 specifically mediates activation of NFATc1, but not NF-κB (Round et al., “Scaffold Protein Dlgh1 Coordinates Alternative p38 Kinase Activation, Directing T Cell Receptor Signals Toward NFAT but Not NF-kappaB Transcription Factors,” Nat. Immunol. 8:154-161 (2007), which is hereby incorporated by reference in its entirety), suggests that Dlgh1-silenced CD4⁺CD25⁻ T cells can respond to TCR stimulation through NF-κB-dependent signaling.

It is notable that two members of the MAGUK family, Carmal and Dlgh1, provide scaffolding for apparently independent, but opposing, signaling pathways in Tregs. PKC-θ mediates negative feedback through Carmal, which is intact in RA patients (Zanin-Zhorov et al., “Protein Kinase C-Theta Mediates Negative Feedback on Regulatory T Cell Function,” Science 328:372-376 (2010), which is hereby incorporated by reference in its entirety). Moreover, Dlgh1 recruitment to the IS in Tregs from RA patients is impaired (FIG. 2A), consistent with their decreased suppressive function, suggesting that regulation of Dlgh1 recruitment may be a primary defect in RA Tregs. T cells from RA patients display higher levels of ERK phosphorylation compared with healthy controls, leading to a delay in tyrosine-protein phosphatase nonreceptor type 6 (SHP-1) recruitment to the IS and sustained TCR-induced ZAP-70 and NF-κB signaling (Singh et al., “ERK-Dependent T Cell Receptor Threshold Calibration in Rheumatoid Arthritis,” J. Immunol. 183:8258-8267 (2009), which is hereby incorporated by reference in its entirety). Interestingly, Dlgh1 can interact with ezrin and contribute to the negative regulation of ERK signaling pathway (Lasserre et al., “Ezrin Tunes T-Cell Activation by Controlling Dlg1and Micro-Tubule Positioning at the Immunological Synapse,” EMBO J. 29:2301-2314 (2010), which is hereby incorporated by reference in its entirety). Collectively, these results together with published data suggest that the activation of signaling pathways is altered in RA patients, and further analysis is required to evaluate the prognostic value of this knowledge.

In summary, Dlgh1 mediates a key activating signal downstream of the TCRs that operates in opposition to the PKCO/Carmal-mediated negative feedback pathway that the researchers of the present invention recently identified in Tregs (Zanin-Zhorov et al., “Protein Kinase C-Theta Mediates Negative Feedback on Regulatory T Cell Function,” Science 328:372-376 (2010), which is hereby incorporated by reference in its entirety). Unlike the PKC-θ/Carmal system, which is reciprocally an activating pathway for Teff functions, the results of the present invention do not support a reciprocal role of Dlgh1 in inhibition of CD4⁺CD25⁻ T cell functions. Although Dlgh1 has no enzymatic activity that could be targeted by a small molecule, it is conceivable that it could still be targeted therapeutically because an RNAi-mediated suppression generates significant impairment of Treg function, which could be useful in vaccination, in combating chronic infection, and in immunotherapy for cancer (Boissonnas et al., “Foxp3+ T Cells Induce Perforin-Dependent Dendritic Cell Death in Tumor-Draining Lymph Nodes,” Immunity 32:266-278 (2010), which is hereby incorporated by reference in its entirety). This therapy would need to be carefully targeted because of potential off-target effects and the diverse roles of Dlgh1 in cell-cell communication in many organ systems (Peer et al., “Systemic leukocyte-Directed siRNA Delivery Revealing Cyclin D1 as an Anti-Inflammatory Target,” Science 319:627-630 (2008), which is hereby incorporated by reference in its entirety).

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of identifying candidate compounds useful as chemotherapeutics or anti-infective compounds or anti-inflammatory drugs, said method comprising:
providing a plurality of test compounds;
incubating the plurality of test compounds with human Regulatory T (Treg) cells expressing Disc-Large Homolog 1 (Dlgh1) or in which Dlgh1 is suppressed, said Treg cells having an immunological synapse;
identifying test compounds which inhibit Dlgh1 expression, recruitment to the immunological synapse, and/or activity in the Treg cells as candidate compounds potentially useful as chemotherapeutics or anti-infective compounds; and
identifying test compounds which enhance Dlgh1 recruitment to the immunological synapse, and/or activity in the Treg cells as candidate compounds potentially useful as anti-inflammatory drugs.

2. The method of claim 1, wherein candidate compounds that are potentially useful as chemotherapeutics for cancer are identified.

3. The method of claim 1, wherein candidate compounds that are potentially useful as anti-infective compounds are identified.

4. The method of claim 1, wherein candidate compounds that are potentially useful as anti-inflammatory drugs are identified.

5. A method of treating an inflammatory condition in a subject, said method comprising:
selecting a subject with an inflammatory condition and
administering to the selected subject an agent which enhances Dlgh1 expression, recruitment to the immunological synapse, and/or activity under conditions effective to treat the inflammatory condition.

6. The method of claim 5, wherein the subject is selected based on it having an inflammatory condition mediated by Dlgh1 protein expression, recruitment to the immunological synapse, and/or activity under conditions effective to treat the inflammatory condition.

7. The method of claim 5, wherein the subject is human.

8. The method of claim 5, wherein the inflammatory condition is selected from the group consisting of an autoimmune disease, rheumatoid arthritis, pericarditis, vasculitis, lupus, bronchitis, phrenitis, acute and chronic enterocolitis, ulcerative colitis, inflammatory bowel disease, type I diabetes, multiple sclerosis, psoriasis, inflammatory bowel disease, Crohn's disease, ulcerative cholitis, and autoimmune disorders.

9. The method of claim 5, wherein the agent is selected from the group consisting of inhibitors of tumor necrosis factor-α (TNF-α) effects on Dlgh1 recruitment to the immunological synapse and direct activators of p38 mitogen activated kinase action on nuclear factor of activated T cells.

10. The method of claim 5 further comprising:
monitoring progression of the inflammatory condition in the selected subject.

11. The method of claim 10 wherein, when said monitoring indicates progression of the inflammatory condition in the selected subject, said method further comprises administering to the selected subject the agent which enhances Dlgh1 expression, recruitment to the immunological synapse, and/or activity to delay progression of the inflammatory condition.

12. The method of claim 10, wherein said monitoring comprises:
measuring expression, recruitment to the immunological synapse, and/or activity of Dlgh1 in CD4⁺ CD25⁺ regulatory T cells in the subject before and after said administering and
comparing the Dlgh1 expression, recruitment to the immunological synapse, and/or activity before said administering to the Dlgh1 expression, recruitment to the immunological synapse, and/or activity after said administering, wherein progression of the subject's inflammatory condition is determined based on said comparing.

13. The method of claim 12, wherein said measuring further comprises:
contacting a sample of Dlgh1 in CD4⁺ CD25⁺ regulatory T cells from the selected subject with a reagent which recognizes Dlgh1 expression, recruitment to the immunological synapse, and/or activity.

14. The method of claim 12 further comprising:
developing a follow-up treatment regimen based on said comparing.

15. A method of treating cancer in a subject, said method comprising:
selecting a subject with a cancer and
administering to the selected subject an agent which inhibits Dlgh1 protein expression, recruitment to the immunological synapse, and/or activity in CD4⁺ CD25⁺ regulatory T cells under conditions effective to treat the cancer.

16. The method of claim 15, wherein the subject is selected based on it having a cancer mediated by Dlgh1 protein expression, recruitment to the immunological synapse, and/or activity in CD4⁺ CD25⁺ regulatory T cells under conditions effective to treat the cancer.

17. The method of claim 15, wherein the subject is human.

18. The method of claim 15, wherein the cancer is selected from the group consisting of is acute lymphocytic leukemia, acute myelogenous leukemia, biliary cancer, breast cancer, cervical cancer, chronic lymphocytic leukemia, chronic myelogenous leukemia, colorectal cancer, endometrial cancer, esophageal cancer, gastric cancer, head and neck cancer, Hodgkin's lymphoma, lung cancer, medullary thyroid cancer, non-Hodgkin's lymphoma, multiple myeloma, renal cancer, prostate cancer, glial and other brain and spinal cord tumors, pancreatic cancer, melanoma, ovarian cancer, liver cancer, bone cancers, and urinary bladder cancer.

19. The method of claim 15, wherein the agent is selected from the group consisting of tumor necrosis factor-α (TNF-α) and p38 inhibitors acting on CD4⁺ CD25⁺ regulatory T cells.

20. The method of claim 15, wherein the agent reduces or silences Dlgh1 protein expression.

21. The method of claim 15 further comprising:
monitoring progression of the cancer in the selected subject.

22. The method of claim 21 wherein, when said monitoring indicates progression of the cancer in the selected subject, said method further comprises administering to the selected subject the agent which inhibits Dlgh1 protein expression, recruitment to the immunological synapse, and/or activity in CD4⁺ CD25⁺ regulatory T cells to delay progression of the cancer.

23. The method of claim 21, wherein said monitoring comprises:
measuring protein expression, recruitment to the immunological synapse, and/or activity of Dlgh1 in CD4⁺ CD25⁺ regulatory T cells in the selected subject before and after said administering and
comparing the Dlgh1 protein expression, recruitment to the immunological synapse, and/or activity before said administering to the Dlgh1 protein expression, recruitment to the immunological synapse, and/or activity after said administering, wherein progression of the selected subject's cancer is determined based on said comparing.

24. The method of claim 23, wherein said measuring further comprises:
contacting a sample of Dlgh1 from the selected subject with a reagent which recognizes Dlgh1 protein expression, recruitment to the immunological synapse, and/or activity in CD4⁺ CD25⁺ regulatory T cells.

25. The method of claim 23 further comprising:
developing a follow-up treatment regimen based on said comparing.

26. A method of treating an infectious disease in a subject, said method comprising:
selecting a subject with an infectious disease and
administering to the selected subject an agent which inhibits Dlgh1 protein expression and/or activity under conditions effective to treat the infectious disease.

27. The method of claim 26, wherein the subject is selected based on it having an infectious disease mediated by Dlgh1 protein expression and/or activity under conditions effective to treat the infectious disease.

28. The method of claim 26, wherein the subject is human.

29. The method of claim 26, wherein the infectious disease is selected from the group consisting of bacterial, viral, parasitic, fungal, helminthic, and a prion infection.

30. The method of claim 26, wherein the agent is selected from the group consisting of tumor necrosis factor-α (TNF-α) and inhibitors of p38 mitogen activated kinase.

31. The method of claim 26, wherein the agent reduces or silences Dlgh1 protein expression.

32. The method of claim 26 further comprising:
monitoring progression of the infectious disease in the selected subject.

33. The method of claim 32 wherein, when said monitoring indicates progression of the infectious disease in the selected subject, said method further comprises administering to the selected subject the agent which inhibits Dlgh1 expression and/or activity to delay progression of the infectious disease.

34. The method of claim 32, wherein said monitoring comprises:
measuring expression and/or activity of Dlgh1 in the subject before and after said administering and
comparing the Dlgh1 protein expression and/or activity before said administering to the Dlgh1 protein expression and/or activity after said administering, wherein progression of the selected subject's infection is determined based on said comparing.

35. The method of claim 34, wherein said measuring further comprises:
contacting a sample of Dlgh1 from the selected subject with a reagent which recognizes Dlgh1 protein expression and/or activity.

36. The method of claim 34, wherein said measuring further comprises:
developing a follow-up treatment regimen based on said comparing.

37. A method of inhibiting regulatory T cell activity, said method comprising:
administering to the regulatory T cells an agent that inhibits Dlgh1 protein expression under conditions effective to inhibit regulatory T cell activity.

38. The method of claim 37, wherein said administering reduces or silences Dlgh1 protein expression.
